SEQUENCE LISTING (132 sequences)

SEQ ID NO: 1 - DNA, 4325 bp, Homo sapiens
  CDS (73)..(3417): EIF-2 alpha kinase
ggagctccaagcggcgggagaggcaggcgtcagtggctgcgcctccatgcctgcgcgcggggcgggacgctgatggagcgcgccatcagcccggggctgctggtacgggcgctgctgctgctgctgctgctggggctcgcggcaaggacggtggccgcggggcgcgcccgtggcctcccagcgccgacggcggaggcggcgttcggcctcggggcggccgctgctcccacctcagcgacgcgagtaccggcggcgggcgccgtggctgcggccgaggtgactgtggaggacgctgaggcgctgccggcagccgcgggagagcaggagcctcggggtccggaaccagacgatgagacagagttgcgaccgcgcggcaggtcattagtaattatcagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccgagaccgtgaaagcatggaaacagttcctttcacagttgaatcacttcttgaatcttcttataaatttggagatgatgttgttttggttggaggaaaatctctgactacatatggactcagtgcatatagtggaaaggtgaggtatatctgttcagctctgggttgtcgccaatgggatagtgacgaaatggaacaagaggaagacatcctgcttctacagcgtacccaaaaaactgttagagctgtcggacctcgcagtggcaatgagaagtggaatttcagtgttggccactttgaacttcggtatattccagacatggaaacgagagccggatttattgaaagcacctttaagcccaatgagaacacagaagagtctaaaattatttcagatgtggaagaacaggaagctgccataatggacatagtgataaaggtttcggttgctgactggaaagttatggcattcagtaagaagggaggacatctggaatgggagtaccagttttgtactccaattgcatctgcctggttacttaaggatgggaaagtcattcccatcagtctttttgatgatacaagttatacatctaatgatgatgttttagaagatgaagaagacattgtagaagctgccagaggagccacagaaaacagtgtttacttgggaatgtatagaggccagctgtatctgcagtcatcagtcagaatttcagaaaagtttccttcaagtcccaaggctttggaatctgtcactaatgaaaacgcaattattcctttaccaacaatcaaatggaaacccttaattcattctccttccagaactcctgtcttggtaggatctgatgaatttgacaaatgtctcagtaatgataagttttctcatgaagaatatagtaatggtgcactttcaatcttgcagtatccatatgataatggttattatctaccatactacaagagggagaggaacaaacgaagcacacagattacagtcagattcctcgacaacccacattacaacaagaatatccgcaaaaaggatcctgttcttcttttacactggtggaaagaaatagttgcaacgattttgttttgtatcatagcaacaacgtttattgtgcgcaggcttttccatcctcatcctcacaggcaaaggaaggagtctgaaactcagtgtcaaactgaaaataaatatgattctgtaagtggtgaagccaatgacagtagctggaatgacataaaaaactctggatatatatcacgatatctaactgattttgagccaattcagtgcctgggacgtggtggctttggagttgtttttgaagctaaaaacaaagtagatgactgcaattatgctatcaagaggatccgtctccccaatagggaattggctcgggaaaaggtaatgcgagaagttaaagccttagccaagcttgaacacccgggcattgttagatatttcaatgcctggctcgaagcaccaccagagaagtggcaagaaaagatggatgaaatttggctgaaagatgaaagcacagactggccactcagctctcctagcccaatggatgcaccatcagttaaaatacgcagaatggatcctttctctacaaaagaacatattgaaatcatagctccttcaccacaaagaagcaggtctttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcagattcatggaaacagctattctcataaagtggacatcttttctttaggcctgattctatttgaattgctgtatccattcagcactcagatggagagagtcaggaccttaactgatgtaagaaatctcaaatttccaccattatttactcagaaatatccttgtgagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaactaaaatggatatatttttataatgcagaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttttatttttcactgtaaatatgtttatgttttatttataaaaattctgaaatcaatccatg

SEQ ID NO: 2 - protein, 1115 aa, Homo sapiens
MERAISPGLLVRALLLLLLLGLAARTVAAGRARGLPAPTAEAAFGLGAAAAPTSATRVPAAGAVAAAEVTVEDAEALPAAAGEQEPRGPEPDDETELRPRGRSLVIISTLDGRIAALDPENHGKKQWDLDVGSGSLVSSSLSKPEVFGNKMIIPSLDGALFQWDRDRESMETVPFTVESLLESSYKFGDDVVLVGGKSLTTYGLSAYSGKVRYICSALGCRQWDSDEMEQEEDILLLQRTQKTVRAVGPRSGNEKWNFSVGHFELRYIPDMETRAGFIESTFKPNENTEESKIISDVEEQEAAIMDIVIKVSVADWKVMAFSKKGGHLEWEYQFCTPIASAWLLKDGKVIPISLFDDTSYTSNDDVLEDEEDIVEAARGATENSVYLGMYRGQLYLQSSVRISEKFPSSPKALESVTNENAIIPLPTIKWKPLIHSPSRTPVLVGSDEFDKCLSNDKFSHEEYSNGALSILQYPYDNGYYLPYYKRERNKRSTQITVRFLDNPHYNKNIRKKDPVLLLHWWKEIVATILFCIIATTFIVRRLFHPHPHRQRKESETQCQTENKYDSVSGEANDSSWNDIKNSGYISRYLTDFEPIQCLGRGGFGVVFEAKNKVDDCNYAIKRIRLPNRELAREKVMREVKALAKLEHPGIVRYFNAWLEAPPEKWQEKMDEIWLKDESTDWPLSSPSPMDAPSVKIRRMDPFSTKEHIEIIAPSPQRSRSFSVGISCDQTSSSESQFSPLEFSGMDHEDISESVDAAYNLQDSCLTDCDVEDGTMDGNDEGHSFELCPSEASPYVRSRERTSSSIVFEDSGCDNASSKEEPKTNRLHIGNHCANKLTAFKPTSSKSSSEATLSISPPRPTTLSLDLTKNTTEKLQPSSPKVYLYIQMQLCRKENLKDWMNGRCTIEERERSVCLHIFLQIAEAVEFLHSKGLMHRDLKPSNIFFTMDDVVKVGDFGLVTAMDQDEEEQTVLTPMPAYARHTGQVGTKLYMSPEQIHGNSYSHKVDIFSLGLILFELLYPFSTQMERVRTLTDVRNLKFPPLFTQKYPCEYVMVQDMLSPSPMERPEAINIIENAVFEDLDFPGKTVLRQRSRSLSSSGTKHSRQSNNSHSPLPSN

SEQ ID NO: 3 - DNA, 4116 bp, Homo sapiens
  EIF-2 alpha kinase, PEK-PRO5
tcttggttgttttgggcaaccctggctcagggtacctgagcaccagcttcttttcctggcccagcctcacgggccagctctcatgcccggtccccactttcttacatttccctgaggcacccaggttccagagttcccacaaagtcactgtgaagctccatgctgtcctaaagcaggtagactctcttttctctccttaatttattttcccagtcagcacacttcgactcaggctttttttccaaaatggaaaatttgtgttttgttcccaagtataaagcttgactctccttactggcattttccaccactgtgctcttctgcccgcgcctttttcatcatagcacttatctcagttttaataatatatgtgtgattgttaatgtctgtctccctaactagataggtgttaaccttcagaagggcgggaaccacatctattttgttcatatctttattctcattatttgcaggtggtcatgtggaatccctgaatgttaaatgaataaataaaacttccacagtatttacaggtggcaagtagactcctaattcattagttcagattaatagccttgttcatgccatgatcattttttgaaaaaattacaaaaccatgaagaccaggcccactaaaatatatacactaatttcaaacaggtagttaacaatcatttttcatcttatgttaattttctgacaccttcctcataccaactagtataatcccttttaggtgggcaattttatctcctattttgttgagtagataatggtcaattggtttgagttcgctcatctattttctctacttttcaaaataacttactctctaggaatacctcctacgctctactttcaacatggacggcagagctgccccttttcctccagatactctggaatcttgctctcgtaatcaaccccctctgtctacagcaactgcagtctcttcctttccagttgtctctttccttctgtcctcaggtattcattcattcattcattcattcaacaaacatttattaaatgcttgctaaacactttgcactgttttatgtcttcgaatacatcaatgagcaaatcttcacagaatttacatgcaagtggaaggaaacacagcagacaataaacaaaacaaatatgtaaattacagtatttacatatttgtaatgtatgtatcggctaagcagaaataaagcaggagagaaataaaggaaggaaggtggtggaggttttaattttaaataaggtagtgaggagggacttcactgggattagcaaagcctcaaataaagtgagggaacatatcttgtgggtacctgggtattatgtgctaaattgtgtccctccaaaatttacatgttgaagtcccaacctttagtgcttcagaatataactatactgtatttggagataagatctttaaagcagtgattaagttaaaatgaggctgttaaaggtagccccacactccaatctgactggtgttagaagaataggaagagacatcagagggaagaggaagtaagagggctgtcatcggcaagccaaggagagaggcctcagaatcccaccttgatcttggacttctagcctccagaacttcgagaaaataaactagttttgttcaggccacctggtctgtggtatttgttagggcaactctagcaaactcatatacctgggaagtttcctaggcaggcaaaacaacaaaggaaaacccccaaggtgggtcttgattggcccagtggagtgagcaaagacagtatgaaatgagatcagaaaagccataggaaccagatgctgtagcaccctgtagtctattttaaggacttgacttttatcctgagtgaactgggggaccttttgagggttacgaccagcactatggagaagcaagaagaccggtgaaaggtgttctagacaggaaaaacttgagttgctggatcaaatacagctgataaaacaaagattatcctttgaatacagcactggtggcctcagcgagagcagttttggtggtgtgaatgcctgactgtagtgtacttgggaatgggagaagaggaattgaagataaggaattttgaacacttgagttttgccatacagaggagcaaagacacgtggtaggagctggaaggggaagagaggtttcttgtttgttttgggctggggagagattagagcatgtttttagcagatgaaggataatctaggtatacccatattgccaacaccttaacaaatcttcaccggttttggaccaggtgcggtggctaatgcccgtaaattgcagcactttgggaggctgaggcgggaggatggcttgaggccaggaatttgagaccaacctgggcaacacagaaaccccatctttacaaaacaaaattaaaaattggcctggcatggtggcacgcatctgtagtcccagctactccggaagctgaggaaggaagatcgcttgaacgcaggaattcaaggttatagagaactatggtcaagccactgcactccagcctgggcaaaagagcaagaccctctctctaaaaaaaaaattttttttaatgttcactggttttgatgcgggtaatagcctcccggccagtctcctcctcgttgatgctgtcactgctgaagatgcattttcttatcacttcactcatctgctcaaaaatcttcaggaagtcttgacttgcagcattaaaagttcaaatgccttggctgaacattccagtcttctccactctgcccttttgcaatttcatcactgtctttggtgaggtacaaagcgtgccaggtcagagtcagaagacatgaattaaagtcatgattctgtgaccaaccagctacgtgatcttaggctaactacgtcagtgcactgggccggggctccttcccgttcctaaggaggcggaggcgtgtcgggcagactggattgtcacaggtcactgccatctctaacaagcggacttctgagccccgttgcggccacaagtaggaccatctcctgcatcctccttacccttctacttctagggaccacacggcttctgtggccacttcttgctgcttcgcttttgccttcctaagtagactaggctttagaagagctacaataccagctcctttaaggtcgacctcctcccggtcacaaggcacttgcctcccactcttcacttgggacagtcctcttcacagtcagaatccgccacgtagtaagtgccgcttccaaccaatcaagaggcagttagcgcagacctttgagggacatccacttccaccaatgatcttcaagtcttctccagcgcctcgctttgtggggcgaggccaaccaccgcgatggccaatctgttgtaggaaaggtattccgggaactgatgagcgcaccaatcaggtaaaaagacgtcggggaagggcatttctcattggtaattgcgtccggaagagggacgggcctcgaacgacgaaattacgatttgattggtaggtgcgatgttgaccaccagggaaagtccaccttccccaacaaggccagcctgggaacatggagtggcagcggccgcagccaatgagagagcaaacgcgcggaaagtttgctcaatgggcgatgtccgagataggctgtcactcaggtggcagcggcagaggccgggctgagacgtggccaggggaacacggctggctgtccaggccgtcggggcggcagtagggtccctagcacgtccttgccttcttgggagctccaagcggcgggagaggcaggcgtcagtggctgcgcctccatgcctgcgcgcggggcgggacgctgatggagcgcgccatcagcccggggctgctggtacgggcgctgctgctgctgctgctgctggggctcgcggcaaggacggtggccgcggggcgcgcccgtggcctcccagcgccgacggcggaggcggcgttcggcctcggggcggccgctgctcccacctcagcgacgcgagtaccggcggcgggcgccgtggctgcggccgaggtgactgtggaggacgctgaggcgctgccggcagccgcgggagagcaggagcctcggggtccggaaccagacgatgagacagagttgcgaccgcgcggcag

SEQ ID NO: 4 - DNA, 612 bp, Homo sapiens
  EIF-2 alpha kinase, PEK-ex1
ggagctccaagcggcgggagaggcaggcgtcagtggctgcgcctccatgcctgcgcgcggggcgggacgctgatggagcgcgccatcagcccggggctgctggtacgggcgctgctgctgctgctgctgctggggctcgcggcaaggacggtggccgcggggcgcgcccgtggcctcccagcgccgacggcggaggcggcgttcggcctcggggcggccgctgctcccacctcagcgacgcgagtaccggcggcgggcgccgtggctgcggccgaggtgactgtggaggacgctgaggcgctgccggcagccgcgggagagcaggagcctcggggtccggaaccagacgatgagacagagttgcgaccgcgcggcaggtgaggggctgccgacccgggggaggcaacttgtttacgcgcgcgagccgcggaggatgcggtgtangggggcggagatccgggacccgggcgggcgtcttccctcggctgcggagggcagctggcgacctggggaggagcgcggggccacgacgccctcccatcccccggccagcgacctgcctgggctcggctcccgagggcctggtgctggccgacgggtcagagcagcatc

SEQ ID NO: 5 - DNA, 1896 bp, Homo sapiens
  EIF-2 alpha kinase, PEK-ex2
aacatggtatgtttctcttcagatacgttcaactgcacagatgtaggagtgagaaaggggagaagattgactttaaccagttcttccagagtgtgacatgtgagaggcagggtagggagttgaggtgtgtgcaaagtagtgcttaagatgaaggactgtgggattttaactggttaagaaagaagtgagggcatggtggtagtgaaggttgtagtatcagtggcttgtaggttctcatagggtcagaagtttcttggagtcagggaagtagaggaagtgagctgaaaagagaggggttggtggttagggggttgcggtgatttgtaatgacaaggtctagagtctgaccacaggagcagctgaagcaaggtagatagatattgaaaacccaaagaattgaggcagaagtatgttaaatatgttaggtggtgacagtaaaccagcagctgaaatcctccaggatggggctagttacctaagggtcaattagatgtctactaggaggggtaagggacaaaacagtctgatcctgaggattttcagagaggagaggaaaaaagtggtctggaaatggcaatgagatacatggagtccacttaccccattctgagcaagggttatgggagaaaaaaattatctgtgcttctgtagggaagcgatatcctcagggaaagcccggtttctatgagagcaaaaagataagtgaacgatcagggaagacagtgtttcaggggaaaggctttcaggaggtatgtaggtagaagaggacataccaggggacattgtgttccttatgggaattagagtgtggaatgaagggtgacctatgagtcaggggcttttcataagttacataaacagataaagggcatattgaaattgtcttggtccaaagacagtggtggcaagagtggtagaaggccccctcttcctttctacgaatagaggcctggacatgggctggttttcttttgagcatgtgggataagtgcccaatatcttagatatctgttaattttaaaaatatttttaatatttacaggtcattagtaattatcagcactttagatgggagaattgctgccttggatcctgaaaatcatggtaaaaagcagtgggatttggatgtgggatccggttccttggtgtcatccagccttagcaaaccagaggtaagaattttctgttaactgttgactagaaaacttaattctaatgagtaattgctgatattaagaagtttggggccctattgcccaggtttgaggcctgtctgcctcttacagtttgtgtccctttagggcaattacttaacttttctattcctcagttcccctcttgtgaaatgagatggataataacatcttcttaggattacttggggcattaagtgagttaatcctataagtgagcagctgaggatactatctgccatatcagcaaagcacattatctgagctataaatgattgtttattatcatcacaagatctctaggaataataagataaatataaataaaaactttattgaattttactagttagaaatctgtgctgcaaaatcccataaattattattccctaattataagcagaattcatctgaaatttttttgtaatgtaattccaggtaaatttgattatttgcagaaaaagtgtacttaataatttggagctctagaatagtagaggggaaagagcatagattcaaagagacctcgcttccaaaattactctgtcacatgacttcaagcccctcagagctttagttgttctcatcaataaagtgaggacacagcctgcccgcatggggtacagatgggggagattaaataagagaagctgattttctcaccttagtttcaattctgatggataagtctctctgcttttct

SEQ ID NO: 6 - DNA, 1595 bp, Homo sapiens
  EIF-2 alpha kinase, PEK-ex3
tgcccaaacgttactttctcagtggggtctaccctaagctctttattgataatcccctctccaccccatgcgtatttaccattctccctttatcttgttccatttttttaacggcatttatcacctaacataccatataatttacttatttattaagttgtttgtctcgtgacaccagagtttaaccttcacaaaggcagtgatttgtttgctttgctcgctaatctatcccaaccatcagaatgtgccaggccataggnaagcccttaataagcattgttaaaagaagggagggcaaccgagaacacaaaaccagtaagcttacttactaggcttctatctcattgcagatttgagaaagcaaatgaaagaagggatgggacactacttgaatcagtatctctaaacctgtgttccttggagctgagtctatgacagtaattggccttgataaaaatagccctagaaaatactgcatatattatctatttacacattaaatattcatattacacatattacacataatcatgttacacattaacatactaattgctataagagctcctatagtaacctcttcttgaactcacttgatcataaaactctcttttggtaactcacctaccattatgggacccttttggcccatggtaaactgagtttgagaaaaatcttactagagtactatgtgtagggcctgggagtgattggcagttcttttaaattacttttggttgatggactgcactgcttcatgtgctactcagaaggaggctggagtacatcaggatcaagactccagctcttaattactattattcttttaaaggtatgatgcttctatttttctgggagaaataagaaaaaaataataattaatgttatggcccttttaaaaagttagcttctgttttaggtatttgggaataagatgatcattccttccctggatggagccctcttccagtgggaccgagaccgtgaaagcatggaaacagttcctttcacagttgaatcacttcttgaatcttcttataaatttggagatgatgttgttttggttggaggaaaatctctgactacatatggactcagtgcatatagtggaaaggtaagtgaaaatgctgaatttactttggggaaatcagagtaaattagggtagaaaaagtaatttattaaactacacttattattagttgagttttattgtaattttcccctgaggttgtcatttgttttaataagagaactgtgaggtaggaaggggaaactaataacagaataaatggcagagccaggaatagcaggaggaagagaattcataaatatggtctactgtgtctcaggggggattttttttttttttttttttgagacagagtctcactctgttgcccaggctgatctcagctcatggcaatccccacccccaccccattccacaccccctcangttcaagtgggttcaagcgattcttgtgcctcagcctcctgagtagctaggattacaggcacatgccaccatgcttggctaatttttgtattcttagtagagacagggttttactgtattgccaggctggtctccagc

SEQ ID NO: 7 - DNA, 1257 bp, Homo sapiens
  EIF-2 alpha kinase, PEK-ex4
ggtagtctcatctgtaaacaacaggattgaacccgatcacctggttttccgatttgatgtgctgctacataattctggtattgtagaaggtatgcttttcgtgaggattttagtttggatcatattaactcttccttttttctttagtgaaaatttgaggcagttacttttgaatacaaaaagctctcagaaaagtttcaaatttttaaaaaccaaacacttttgttatacagaaactctaaggttgattttttttttaactcacctgaaattttattaatgatattgtagaaaagctatcacaagtagctatccatttcttcttgtatattctatggaaatctccaaagtaaggctaaaattatgtaaatccttaaaatcattccctgaaataaatattcattggtactgtcattgttctaaataactcattttaggagttggtaatctaactgatgcttcttatgacttgagtacttcatacacatttcnttagtttcctttcacttttttaaaaattacagattcctttaatatctctgatctattatgagttgtctccttttactaattttgtatctaattttgtcttttcaggtgaggtgaggtatatctgttcagctctgggttgtcgccaatgggatagtgacgaaatggaacaagaggaagacatcctgcttctacagcgtacccaaaaaactgttagagctgtcggacctcgcagtggcaatgagaagtgtgtattcagataatgttgctgttggtattatttagaaatacacctaataccaaaatttatcagatttctgtttgtggagattttgactattttgttgccttaaaagcatatatatatatatttttttgatacggagtcttgctctgtcgcccaggcttgagtacagtggcgtgatattggctcactgcaacctccacctcctgggttcaagcgaatctcctgcctctgcctcccgagtaccggggattacaggcacgtgccaccacacccaactaatttttgtatttttagtagagacggggtttcaccatgttggccaggctggtgtccaactcccgacgtcaggtgatccaatgtgggtcctaaaataaaaatggtttcatggttattaacaaattctgaactgaacttctcaccatatgcttcgggatatgataaccacagggnnnnnnnnnnnnnnnnnnnnnnnnnnnnnnnnnaaggttaaatggatttttttttttttttttttttttttt

SEQ ID NO: 8 - DNA, 4375 bp, Homo sapiens
  EIF-2 alpha kinase, PEK-ex5-8
aagatccaagattggggctgaggtatcctggatcacattgcctttttgaggccatgttttagtatgaatttaagactggtgcattcatcttgacttttacactggtttgttagggcatattaattttgctgcacttaatgaaatgtatcctgcctttaattaagaggtaaggcagactgtgtgctacctcattttaaggtgacattgatgtgtttggggaaaatcactctgatgtagaagtacaaacatttgtaagtttttgagagaaaatctgtcccttagctgttgtaagggacacatcaagtcagtccacaaccctcaaaaccattgtgtctgaagggtcaggacaaagttcttgtgggccctcttgtggcataaatcagtagagcactcttttccagaaggttatgttgttagttttctcatcacataattttagtatttgcttcttcaatctagaagagttctatattattttgtccctttctttaaatgtaaatttctaaaacacacctttgtaaatttaaggtggaatttcagtgttggccactttgaacttcggtatattccagacatggaaacgagagccggatttattgaaagcacctttaagcccaatgagaacacagaagagtctaaaattatttcagatgtggaagaacaggaagctgccataatggacatagtgataaaggtttcggttgctgactggaaagttatggcattcagtaagaagggaggacatctggaatgggagtaccaggtacctaacaccactgaggatttaaaatacggttcttcctctcccagtctgaccaaacttattgattgggtggaacgaaattactactacttggggctctcagcttgttctctgtgcttttataaatttgtgattttaaatggtattttatgggttggaactatataactactgcttgaattatttaagaccttttttccatttttgtttagttttgtactccaattgcatctgcctggttacttaaggatgggaaagtcattcccatcagtctttttgatgatacaagttatacatctaatgatgatgttttagaagatgaagaagacattgtagaagctgccagaggagccacagaaaacagtgtttacttgggtgagtaaatgtatcttatctaacgatagtacacattgacatctagattttcttcttacattgttccttcctacttcaggagtgcctgtagtagttttaaatcctaatatcatctctgatgtacgttgcccttgagatttatacttcgatttccattcctgctacttttccatttgtccaattctgaaaatttttttgttgttgttggagatggagtttcactcttgtcgcccaggctagagtgtgatggcatgatctctgctcactgcaacctccgcctcctgggttcaagcgattctcctgcctcagcctcctaagtagctgggattactggcacctgccaccatgcccagctaatttttgtatttttagtagagatgggatttcaccacattggccagaatatagtgcacctgacctcaggtgatccacccacctcggcctcccaaagtgcngggattacaggcatgagccaccacgcccagcccaattctgaaatttttaattactgtcgcatattctttttctctgaggtctatattagaaaggcttagaaatattctcattatataacatgatttcaaattactcattagccaagaatggtggcacgcacctgtaatcctagctactccagaggctgagatgggaggatcgcttgaccccaggagttagagcctaccccaaactataatcatgtcactgcactcctgcctgggtgacaggacaacactctgtctgtttatatatatataattatttataagtaattacatatatgtattacatatataattatttatatgtaattacatataaaatatatagttaggtatatatcctcaaagtctataattttctgtatttgtatttgcatccatattagtttgtgaccctcccctctttaagattagatttctttcttttttatcagtgcatctgtacaactgatctaaaaaataaaactctggtgtgcttctgggcaattgaaagcctttaatattataaattttgaaaactcttggatctaatttgaactagtctgcatcatcaaatactcataaaattctataagctctgacaatgtgccatccacctgttggtttgaatgaaaagcaagaatttgaaggaataacatgtcatttgtgttatgatagtatttaactgaattgttagcaatatttctagaattataggtgtttaggtaaactttcttgagaaagttacttagtgtaagctatttgttttgtgagagtacagtgacttatctttgaatttttctactggaacaattttcctgtattactgaaaatgtgctattattcagtgagaaatatcatatggaccttttgtgtaactttccctccctgtttttgttgaataaacattgagtttatctttgagtgcttcaagcatgtttcttctttgacaagagttttgtggtgtatgtagaaataactggaattaatgtaattttatttaattaaaaaaacctttttaaaaaaatcaatgcataattgacaatgttctggttgattcaggaatgtatagaggccagctgtatctgcagtcatcagtcagaatttcagaaaagtttccttcaagtcccaaggctttggaatctgtcactaatgaaaacgcaattattcctttaccaacaatcaaatggaaacccttaattcgtaagtgaattgtaaacttttctaaatactgttagtgttcagagacctaatcctgactgtctttgccctagttttgaatactgcagagataagaactgttatatactttatattttattgataaaccatgatggtatttcagatgttaataatgaatttttattttcatttagagcatcatttcttggaagcagcagttgcttctaaaaaatgttaatcaaatatttctctatacaacttagaaaaactcttaatcattccctgaccatgccaaagtaacatttagcgcttaacatacttatattaagactgtttaggcaaaagttatctggttagcacatcatattctttgagactgaacgaatagaaatcaaatactgtgcacctacttctttcattatctttctaaaccttgtacgtgttttttactacatatgtcatgtcatattttaattgtttgtttaaacaactcagattctctgtaaaactgtgcttttcaaaggcaagagctctgggccatttgtttaacttatatgtttgaattgaattatggttgaatatcagtttattcaattaagcacgtgcatttttattcaagtcataacagttcagacttaagaaatattattattaagaaaataataattttcttttagattctccttccagaactcctgtcttggtaggatctgatgaatttgacaaatgtctcagtaatgataagttttctcatgaagaatatagtaatggtgcactttcaatcttgcagtatccatatggtaagtgaaaatactgagttttatttattttattttttaatttgaaattaatagaattcaaatgaagaaaagtcgattagagtatagacaataaaacatcttgggagacaatttcatgaatgattactaagcatacaagctccaaagttaggttgccagggttcagatcccatttttgccacatgctaggttgggcacatctaactcccctgtgtctcaatttctttatctgtaaaattagaataataatcctaatatctatgtattgagttgttgtgaggcctaaatgagataacgcaggcaaagtctcagttaacatcatacgtggcacatagtgtcagtaaacattggttctcgttattagctcttatttatcagactatattatgtaggctgtatagttgcctgtataatgaagaaaatgtgtttttcataaaactacatgaaaatgatgcacaatgaggttatcttcttactcagacaagagaaattagtgcaaaagtcaagaataggtgagatttggcatgaaatacattttctatttagtaagcagcagttttttgaagttaggatatattcagatatgaaagccttttaaacagttgtgtaattaagaagtcctcaaatctgtatcaggtaaacacgtagacgactcatcagttaccctagatgttagcactggaaagttattatataaattaaattgattaaaaaaaaatggctctgaactagaaacagcagcctttatcttttttttttttttttttttttttttt

SEQ ID NO: 9 - DNA, 1243 bp, Homo sapiens
  EIF-2 alpha kinase, PEK-ex9
aaaaaaaaaaaaaaaaaaggcaagataaaagagaactgtgtagtctgaccacgtagtacaaaatagaagacaaaaaaagncnagctatttttctgggacaaactcattttgacagcctaaactgaaccaaacagcatggatgttttcctttcattttgtgaagaatgattggtggtaaaatttggtattttattgataactaaacaaaaagaaagctaaaaataccctgaaggaagattgagtattaattcttatatttaaagaattgaattattaatgaggttatgaatggagtagcccttaagatttttttctagtcttattacttaatataaagaaaatttaatatgcttataggataaaggaaaatgtctatatttacgggagaaaaatgagacaaattaagatgtttaaaatacgttaaagaagagagacaaaacttaaaaggaattaatgtgataagtcacaggaaaatggataaattttaatagttaaagacgggcctatttttgattacctttaaaaaaaacgttttaatgtttctatttgaagataatggttattatctaccatactacaagagggagaggaacaaacgaagcacacagattacagtcagattcctcgacaacccacattacaacaagaatatccgcaaaaaggatcctgttcttcttttacactggtggaaagaaatagttgcaacgattttgttttgtatcatagcaacaacgtttattgtgcgcaggcttttccatcctcatcctcacagggtaagaatcatggttgcttactgtctggtttccacttccccacctcctatttgcttctcaacccatcacagtctggcttccatccctactgctccaccaaagctactcttgccaaagttctcagtgatcttccttgtgttaaatgtaatggacatttttcagtccttatctaactgaacctctttgtatttgacactgttgaacatctccctttgacctttcttgcctgacttccttgacaccatgctctcctggtctccttgggtgtgatgtggatgcttcaggacctgatctttctcatcctctcagtattggtggtattcctcagcactccctcttttcactgttcatcccacaaactctccttagatggtcttccctactttcccagctccaatcttcttatatactaatgggtcccaaatctgtttctctggaacagctatctagtaagtgctacacangcactt

SEQ ID NO: 10 - DNA, 1608 bp, Homo sapiens
  EIF-2 alpha kinase, PEK-ex10
tagactgtacattcttaaggacaagaaccagtctacactgtttaccaccatctcctcagccccttacacagtgcttggcacataattggagctcagtaaagatttgttgaaagaatcagtgactaaacaggtgacccacagtgccccaatttgaccatgataattattaacatcctctaaaacatcttttaggagatgttggtaaagaggcattgcctgatttaatttcctgttcttaaaagcattttaatgcaatctatgaaactggttaggaaggaaatctctctagtctttttgttgttgttgttattgttactgggttttttgtttgtttgctttttttccctaccttattttgtcaaaagaaatcactctagtcttgcccaggagtttgtgtttatgcttacatgtgtgcattgtctttctatctagttatgtatcatgctgtacatatgacatacccacattataaagaaacaaaatcccctcaaagactggagggatagcagtgggaagataaactttttttcttttataataaagcaaaatgctgcactttgttttcataatgcttttatttttcttgatgctacttatgtatttttcagtgttgtttattttataacctaaaattgttagctaacttcagttcagctttgtactggtagtgattttgtttttcaccttatcagcaaaggaaggagtctgaaactcagtgtcaaactgaaaataaatatgattctgtaagtggtgaagccaatgacagtagctggaatgacataaaaaactctggatatatatcacggtaagagtcttataaaatacaaccatctgaatcaaagaagaaatgacctaagatcttgtttaactttttttttaatgtgtggatatctagaaaaataaaacataggcttaaccctcaataaataaataaattcaggtaacttaaatgtattaaaagtggtatataccctaagaaagataaaaatagagtgttataggaatttagaatttcagctaccaaattaagttcttattcaagtaacttagttatttagggtctactatgtactaggattagcatttatagtaccagataataattaggtttataagagtgtgatatgagtctccacgtttcggaatcctcatttattttcattattgttctatctgtaataggaagtagaaatatagggaaaaaaacactaatagaacagatagtttttaaaagcagaagggggagatggattagctaaaagtaggcagtttaaataaaagtaaagaagctattagcaatctctcaagtataaaatgtcacctttgatgcattgtgataactggaaatggtgttatggtttatttttcatattacatggattatacctatttttctctttgtcttgatagcatacttcttatcactttagtgatatggggacaangaaaagacgtggaactatactgcttaatatctagggtaatgattttatggcagaaaagattgaaaataatagataaatatgtatattggggccctccaaggaaacagaaatgacaagatgtgcatatatatcttatacataggcatatatcttatatatatac

SEQ ID NO: 11 - DNA, 1295 bp, Homo sapiens
  EIF-2 alpha kinase, PAK-ex11
cttgaattgtgagacagcatggtgcaaattgactggatgatacagtgcaggtcagtgaagtcagcactaaagggagaaacaggctgcttagtggaggcccctgtgagtgacagatggtgagttggcttctgtttgacttggtacctccagtggtaggtacactgactcgtggggcaacccaacccatgtcagctttggttcctaggaaggtttatggctaaaatagtacctgggtataataggactgattaaaattttcccataaaattgacaggtaattagctaagataaaaacacattttctgtaattgattacaaaatgtcacagaatgtaaaagatatgaggattaggaatatgatattttggtagatgataggaagtattggacagcacacatcactagtgcagcagttgtaagaaaaagtgattaacaagtgattcccaattaaacatgtcttttttatttttaatttttttctaggaaacataagaatgtgtttgcttcatttatacaaacaggactaaaaatgctgttaatcaaattcaaaatatactatttattaagatgagttctatgagtttatacatttttatgtgtcataagattgaactgattttcacattaccacaaaatttaaaactgttgcaaacctttataaattttatctctttttaaagatatctaactgattttgagccaattcagtgcctgggacgtggtggctttggagttgtttttgaagctaaaaacaaagtagatgactgcaattatgctatcaagaggatccgtctccccaataggtaatgggtggtaccttcagtaaacttgaaatcagcacagtgtgatctaatctcatgggtaaaatatccttcttactgtactctgtaaacaccatagaaaacagtttcagacgtttcaaatcttaggttctaagtgctgccaattaaccagctgtctaaaagttgttatctctataggttgctttctatctacttttaaaatatgcccttgttttttattattaactcaggactttcgtttagtggcttataataactgtagaccagtaaattgcagcattttaaaacattctatatttttgtataaataaggaaaagtactagaacaaaaacatttgaattatattgtctctacctggtaataactattaacactttagagtatttccttttgattttgttttctggtcatatatttacctaactggtagccagttgccagtactgtacagttttgtctgttgctttcttcatcatatcctctatatttttt

SEQ ID NO: 12 - DNA, 3794 bp, Homo sapiens
  EIF-2 alpha kinase, PAK-ex12-13
ccctgtctcaaaaaaaaaaaaaagtataacctaggctaactcatctgtttctaattttagtttcaagaaaagatcctattttattatttttctgttttcactattagatatgaatacttcagatatgaacagccttcagggttgtcttactttctctctttttcaggctaattttatttctttaattttccttttttattggtatataatacatctacatattttaggggtataagtcatataatttgtaaagatcaaatcagtgtaattggaatatccatcaccttaaatattttctctctttcagggaattggctcgggaaaaggtaatgcgagaagttaaagccttagccaagcttgaacacccgggcattgttagatatttcaatgcctggctcgaagcaccaccagagaagtggcaagaaaagatggatgaaatttggctgaaagatgaaaggtaactaactttgttacacatacacttaagctagttttttgcttgtgtgattacaatgtcagttttataactttagggatttttttttttaaagaaaaggaacagcagagttctgttgtttcatgttttgaaaagttctctagccacttgtgaaattttggtttagattttgagaacatacacgggtgactcatgcctgtaatcccagcactttgggaggccgaggtgggaagatggcttgagcccaggagttcaagaccaacctgagcaacatagtgagaccctgtctcttagaaaaaataagagagaacttacattttaaaaaattactagttgataggactctatacattgtgattgattgagggatttatagtgacttttctcagtataaggtatctgcttctgtctccattttttaaaaatgtttgttatttagttctcttagcagttaacaatttacagctcctttttaagtagttttgaactatttgcagttaaaaatacataaacttagcctgaatatattgtccatattaactatgatgtgctataggaaggaaggccctttaaaaactattaaaggagaagaaaaaggaagcatgtgtagatagactgccaccaaacaactggagtgaaactcctacctacaggtatctaagcaacattagatccacgtgaggtaccttgttaaagtggtgcttattatagactcaaccaataactaagccatagaaggaccaagtgaagtggccccatgtctcaaggccttgtgaggcgctgcccttttaggttaattttaagccaccaaggaatcctgtcctcactttgcataaaaggctttggctgctctggcagccccagacagtcccaggtaactgtttcatgtataaaattaagctttaaaattaagttttttagaaggaatgaatggaatgtgatgttctgtatctcacattgcatgtttttatttagtttgtatgttgttttgttgtcattnggtaagtggccttattacagttgaagctttttaaacagagggtgcagttcaggtacttgaatcaatatatattcactcttacccctttgtatttctcccacttttagcacagactggccactcagctctcctagcccaatggatgcaccatcagttaaaatacgcagaatggatcctttctctacaaaagaacatattgaaatcatagctccttcaccacaaagaagcaggtctttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaaggtctgtatttgtggagcatcacccttggggtttcaatctgacgttttgtgattcagagcagtacttgcagtactctgaaggatccttaagagttggggagagtaaaagcatctgagagcagaggtctgagaaagtagcctcgaaggggcctgctgcaagaataagaagtcttatgtctgaaaactttaggcaaaccatgcattcattgtcttcagtaatgtgtttgtgttcattttactgtaaaaggtattctcagtagtccaggtgagggaaaaaaaagaaaaaagaatctttaggtaaaatccaccatgagcagatatagcctgtttttttgtttgtttgtttgtttttgtttttctatggttttgagtaaccctgacaccaatacctccagggctctgaagcagcatggtgaaagggatccgaatggaaggagagacatgggttccttcattagccagcttgaattggggcaaatctccacacttttgcttctttttctgaactgtttagactttgggggaaggggtaagaagccgaatggggaaaggtagcaaatagttagcagatgactgtttacagctctaaaaccttggattctattttcaatatattcagtagtgaattttgcagtaatataatatgcaaaattattaaagagtcttactaaaacatgacatttccgtagtagtgtttctaaaaataagtacatggaacttttatttaactaatttcccacattccatatactcttagccatcaccagtagatgtggagtgaatgtataaatacttttctgatgaaagtagcttaaagtcttgatagatgtggatccattttaagtcttttcagacttaaaacagacttgtttcaggcacaaaagtagctatttggacacaagttattttcttctaaaatcagcagtaggttttcaaattcttgggtatattttaagagttttagggtaacaagaataggaattagaaataattcttattttttaatataattgttatttagtcatataaatcattatgcttcagtgattttgacgtgggcccaaactgattgccaagaattgttctgcactgaatgaaaagttcagagatgaattatttggttggattggagaagacagtttacaggagaccacacacccctaatttgagagaatactagcagttaggtttgaatatagtaaacgcttatcactaggtgggaaacagctagctgaaatacacattcttcttgtacttaacacttgctgtacacacaaatgccaacttgaaagacaatgaatcatgttttcactaataatgaatattcttctgctaattttataatgtaaatgagatattggtaaatatccatttaatgctacaatgttgtctaaagatttttctgtaattgtctatgcaccagaaaaattgcaagaagaaagttaatatt

SEQ ID NO: 13 - DNA, 828 bp, Homo sapiens
  EIF-2 alpha kinase, PAK-ex14
gattgaggattaagtaccatgatccctagggaatgtgcacctcaaagggtttaagtagatgttcaataaatactagcactctttgctacccttcccttttctttactagcagtacttgtctggcacagaaaaattgtcactattttcctgttagcccattttaaaaagaaatatgcttgaaaatatctagtttgttgtattttttctttgtagtcatttaaataattctctttactttttcgcctccatgcacacccactgtacttttgtctgttgtattctttccagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcaggtgagtttttcagacctttacttactagcacagcagcagatgtacctgatgaatctcttctcatgttttcattaaaatacccgttaatctaaaacccaataagtctgaaaattatgaaaactggcagtagtgttccagtagtggaatagtgacacagctaagatgtagtttctacaacctgaattggggctggattaagaaaaatacaacacataaaatgcactcaccccctgaacaagcacctgacagcaaccaggaatttggaaagagactttgaatgccaccctcccaaacttaactcgtttgccttgtgtagactgtgttggaactgntttcagagccagctagacccagaggtactatactgagctg

SEQ ID NO: 14 - DNA, 1222 bp, Homo sapiens
  EIF-2 alpha kinase, PAK-ex15
gagtcaccaaaaggtcaattttaatttaaataatgctttctttagtcgagtagttctgttgtaattcatttgttcattttaacaactaagtatttattgagggccttctctatacttagccccagtgctggacgatagcaacacaaaagaagcatttcccttcctccaggacttgataatctagttgtgaagaataagaaatatacacatgaaaaatcaccaagaatgcaaagtatccgtaattaagtgccctaatgaggctactacagttgattagtggttggagtaatgaagatttctcataaaatggttaggactagatgattagaatgaagatttgcttagatagcgtccttgaaaacctcacctaggggtagtcgagcagattttgctggctcccacaaacttttttagcatggcaagtctccaggtttccgaggggcctgttttactgatgcatatctcagagctatacctgggctttccttctgtaatgctgagtagtttaattactcttggtgtagtagtgaagaaaagagacttgggagattaactctttgctaacatttttacacatgnncntgcatttaaaccaagaagtgactaagtaaactttgggattcaataatgctgtaatatcanctgtactgtctgctgtgttaatttttaaattttctttatgtgggatttcagattcatggaaacagctattctcataaagtggacatcttttctttaggcctgattctatttgaattgctgtatccattcagcactcagatggagagagtcagggtaagtaccctccctactcaaaaaaaaagtttcaaacagaaaataatctaacatttacaaaagagttttttaaagacttagttcttcttaataccagcagattggttaactaaaagtgaaagaggtgatgtgagtaacacagagaggagttctagactatttgcttccatttaaagctcagtcttcaaaaacttgtttggttagattgtttgttcttagtttttgcttaaattcatcataatttaacaatgttttgcactcagtaagtttgttataagtaaaaatctaggggaaccacaaggtaatgggggccacacactcatatatttaaaagctggcagattaagcataattaggtttctttcctctcaaaataaaccatgaccacagcctttaaagcagatagtactgaaagtctttttttttt

SEQ ID NO: 15 - DNA, 3348 bp, Homo sapiens
  EIF-2 alpha kinase, PEK-ex16-17
aaaaaaaaaaaaaccagtcaaaataaccattttcagaacaccagaaataaaggccctaaaaccttccagaaaggaaaataaaacaggtctaacctaagtaaaggatcaagaatcagatggcatcaaaatttatatagttataaatgcctcagagaatactgtcaagaaagtgaaaagacaaggtggacgtggtagcacatgcctgtagtcccagctacgtgggaagcttgaggcaaaaggaattccttgagaccaggagttcacggctacagtaagctatgattatgcctgcaaatagcctgggcaccatgagaccctatctctaaataaattaattaaatgaaatagaaagtgaaaaggcagctcacagaatgggagaaaatatttgtaaatcctatatccgaaaagtgtctagaattcagaatacataaagaactattacaactcaacaaaaagacaactcattttaaaaaggggcaaagcaatagaactttctccaaagaggataaacaaatggccaataagcatgtgaaaagatgctcaacatcgttgggcattagggaaatgcaaatcaaaaccacaatgagataccacttcagcaccatctagggtatctgtaataaaaaaattaaaaaaaaaggaaatgtcaagtgttggctaggatgtggagaaattggaaccctcatacgttgctggtggaatgtaaaatggtgcagctgctttggaaaacagtctggcagttccttaaacagttaaacatagaattaccatagaatccagtaattctactcctaggtatgtactcaagggaaatgaaaacatagacaaaagcttgtatacaactgttcatatgagcattatttctaatatccaaaagtagaaaccaccaaaatgctcatcagctgatgaatggaaaaacaaagtgtggtattttatacaatgaaaagtattcaaccattacaaggaaggaaatactaacatgtgccgcaacgtagatgaatcttgaaaacatgctgagtgaaaaaagccagacacaaaagtccacttttatatcattcagtttttatgaaatattcagaagaggcaactccatagaaacaaagatttccaatattttctttgtgctttaatatggccctttctctctctctctctcccctttctctttctccctccctccacacatacatatatacacatatatgtaaatgtacatacacacacacacacacacacacacacacacacacacacagtcatcttggtatccacaggggattggttccaggactccctgtggataccaaatctgcagatgctcaagtcccttatataaaatggtgtaatatttgtatagaacctacatatattctgtgtattttaaatcttctgtaaattacagtacctaacataatgtaaattctatgtaaataagtattatactgtattgatgagggaataatgacaagaaaaaaatctgtatctgtccagtacagatgcaaccatctatttttaaattttttaaatattttcattctgtggttggttgaatccttggatgtggaatctgtgggatgtggaagaccagctgtatattttgaggatatttgatggagtgtacatctgtgctcaggaaacatatgtagttattaaatcaggaaaagctattgataaattttccatttgatagatgtacaacctcttagtcattttgttagagtatcaaaaaatattttcatgttgtatgtcaaaataaacttaataagtgatactttttttctttttagaccttaactgatgtaagaaatctcaaatttccaccattatttactcagaaatatccttgtgaggtatgtgtaattctcatcttttatcttctttagaatcatctttagaacgtaagcggtccttagcagtcgactcagtttcccctattttacagatgaggaagccaaaggtctagagaggaaagagacctgctcccaagagcccagaatttgttaaaaccaaacactggagctggggcctggctccttcagcctaatgtccagtgttccacactgtagcacctgtgaaatttatatattgataaatcttgaatttccttaagtaattaagttgaagtgagttagtatgtgttcattttcaaattggaaaaagtcaaaatttatctttcagtattataatgaagggttgcattaaaaaatggactttataacaatatattaatacctacatatacttaagtctgttttactaaaaattgtcatcatgtattttgcaaacttgatgaatctattcccagggtggctcataagagtacatgttacgttcaaagagatttttaaaaaccaagaaaaggtgttggttgctgatctctccataattttttctaattaaaatttctattgagataattgtagatttacatcaattataagatgatttttaaaaatcatatttatgtttcagggatgtgattaaataatcctttataatgtgttagaaaatcaaattacccaaaaattgtccatgtttttttggaatgagggttggcaaactagtgcccacaggtcaaatccagcctgccacctatttttataataaagttctattggaacacagccatgcccattaatttacaaattgtctctggatgcttttgtgcattgacggcagagctgagtagttgtatcagagacttgaaaactcgaatatggctcaaaagcttaaaatatgtacagaaatattttgccagcactgattttaaaaactgtacagtgatcaaacctggccgttttatcacaaaacaatttttatattttcagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaac

SEQ ID NO: 16 - DNA, 21 bp, Artificial Sequence
  Description of Artificial Sequence Primer
ggggcataacctaatttgagc

SEQ ID NO: 17 - DNA, 20 bp, Artificial Sequence
  Description of Artificial Sequence Primer
ggggactttccttcttctgc

SEQ ID NO: 18 - DNA, 18 bp, Artificial Sequence
  Description of Artificial Sequence Primer
ctgactggaaagttatgg

SEQ ID NO: 19 - DNA, 20 bp, Artificial Sequence
  Description of Artificial Sequence Primer
aaaagactgatgggaatgac

SEQ ID NO: 20 - DNA, 25 bp, Homo sapiens
cgcgcggcaggtgaggggctgccga

SEQ ID NO: 21 - DNA, 25 bp, Homo sapiens
ttttaatatttacaggtcattagta

SEQ ID NO: 22 - DNA, 25 bp, Homo sapiens
caaaccagaggtaagaattttctgt

SEQ ID NO: 23 - DNA, 25 bp, Homo sapiens
tagcttctgttttaggtatttggga

SEQ ID NO: 24 - DNA, 25 bp, Homo sapiens
tagtggaaaggtaagtgaaaatgct

SEQ ID NO: 25 - DNA, 25 bp, Homo sapiens
gtcttttcaggtgaggtgaggtata

SEQ ID NO: 26 - DNA, 25 bp, Homo sapiens
gcaatgagaagtgtgtattcagata

SEQ ID NO: 27 - DNA, 25 bp, Homo sapiens
ctttgtaaatttaaggtggaatttc

SEQ ID NO: 28 - DNA, 25 bp, Homo sapiens
ggagtaccaggtacctaacaccact

SEQ ID NO: 29 - DNA, 25 bp, Homo sapiens
tccatttttgtttagttttgtactc

SEQ ID NO: 30 - DNA, 25 bp, Homo sapiens
gtttacttgggtgagtaaatgtatc

SEQ ID NO: 31 - DNA, 25 bp, Homo sapiens
ttctggttgattcaggaatgtatag

SEQ ID NO: 32 - DNA, 25 bp, Homo sapiens
cccttaattcgtaagtgaattgtaa

SEQ ID NO: 33 - DNA, 25 bp, Homo sapiens
ataattttcttttagattctccttc

SEQ ID NO: 34 - DNA, 25 bp, Homo sapiens
tatccatatggtaagtgaaaatact

SEQ ID NO: 35 - DNA, 25 bp, Homo sapiens
tgtttctatttgaagataatggtta

SEQ ID NO: 36 - DNA, 25 bp, Homo sapiens
tcctcacagggtaagaatcatggtt

SEQ ID NO: 37 - DNA, 25 bp, Homo sapiens
ttttcaccttatcagcaaaggaagg

SEQ ID NO: 38 - DNA, 25 bp, Homo sapiens
atatatcacggtaagagtcttataa

SEQ ID NO: 39 - DNA, 25 bp, Homo sapiens
tatctctttttaaagatatctaact

SEQ ID NO: 40 - DNA, 25 bp, Homo sapiens
tccccaataggtaatgggtggtacc

SEQ ID NO: 41 - DNA, 25 bp, Homo sapiens
ttttctctctttcagggaattggct

SEQ ID NO: 42 - DNA, 25 bp, Homo sapiens
aagatgaaaggtaactaactttgtt

SEQ ID NO: 43 - DNA, 25 bp, Homo sapiens
ttctcccacttttagcacagactgg

SEQ ID NO: 44 - DNA, 25 bp, Homo sapiens
ggacctcaaggtctgtatttgtgga

SEQ ID NO: 45 - DNA, 25 bp, Homo sapiens
ttgtattctttccagccatccaaca

SEQ ID NO: 46 - DNA, 25 bp, Homo sapiens
cccagagcaggtgagtttttcagac

SEQ ID NO: 47 - DNA, 25 bp, Homo sapiens
tatgtgggatttcagattcatggaa

SEQ ID NO: 48 - DNA, 25 bp, Homo sapiens
gagagtcagggtaagtaccctccct

SEQ ID NO: 49 - DNA, 25 bp, Homo sapiens
tttttttctttttagaccttaactg

SEQ ID NO: 50 - DNA, 25 bp, Homo sapiens
tccttgtgaggtatgtgtaattctc

SEQ ID NO: 51 - DNA, 25 bp, Homo sapiens
tttttatattttcagtacgtgatgg

SEQ ID NO: 52 - DNA, 18 bp, Artificial Sequence
  Description of Artificial Sequence Primer
gagaggcaggcgtcagtg

SEQ ID NO: 53 - DNA, 20 bp, Artificial Sequence
  Description of Artificial Sequence Primer
tttccatgctttcacggtct

SEQ ID NO: 54 - DNA, 20 bp, Artificial Sequence
  Description of Artificial Sequence Primer
ccagccttagcaaaccagag

SEQ ID NO: 55 - DNA, 20 bp, Artificial Sequence
  Description of Artificial Sequence Primer
ctcccattccagatgtcctc

SEQ ID NO: 56 - DNA, 20 bp, Artificial Sequence
  Description of Artificial Sequence Primer
aaggtttcggttgctgactg

SEQ ID NO: 57 - DNA, 20 bp, Artificial Sequence
  Description of Artificial Sequence Primer
atgtgggttgtcgaggaatc

SEQ ID NO: 58 - DNA, 20 bp, Artificial Sequence
  Description of Artificial Sequence Primer
ggagaggaacaaacgaagca

SEQ ID NO: 59 - DNA, 20 bp, Artificial Sequence
  Description of Artificial Sequence Primer
cattgggctaggagagctga

SEQ ID NO: 60 - DNA, 20 bp, Artificial Sequence
  Description of Artificial Sequence Primer
agactggccactcagctctc

SEQ ID NO: 61 - DNA, 20 bp, Artificial Sequence
  Description of Artificial Sequence Primer
gtgaactgggctggagtttt

SEQ ID NO: 62 - DNA, 20 bp, Artificial Sequence
  Description of Artificial Sequence Primer
tctcctccaagaccaaccac

SEQ ID NO: 63 - DNA, 20 bp, Artificial Sequence
  Description of Artificial Sequence Primer
gcatgtcttgaaccatcacg

SEQ ID NO: 64 - DNA, 20 bp, Artificial Sequence
  Description of Artificial Sequence Primer
ccattcagcactcagatgga

SEQ ID NO: 65 - DNA, 20 bp, Artificial Sequence
  Description of Artificial Sequence Primer
tgcaattttggacaggcata

SEQ ID NO: 66 - DNA, 18 bp, Artificial Sequence
  Description of Artificial Sequence Primer
gagaggcaggcgtcagtg

SEQ ID NO: 67 - DNA, 18 bp, Artificial Sequence
  Description of Artificial Sequence Primer
cgcgcgtaaacaagttgc

SEQ ID NO: 68 - DNA, 20 bp, Artificial Sequence
  Description of Artificial Sequence Primer
tgagcatgtgggataagtgc

SEQ ID NO: 69 - DNA, 20 bp, Artificial Sequence
  Description of Artificial Sequence Primer
tgccctaaagggacacaaac

SEQ ID NO: 70 - DNA, 21 bp, Artificial Sequence
  Description of Artificial Sequence Primer
tcaggatcaagactccagctc

SEQ ID NO: 71 - DNA, 20 bp, Artificial Sequence
  Description of Artificial Sequence Primer
tgacaacctcaggggaaaat

SEQ ID NO: 72 - DNA, 23 bp, Artificial Sequence
  Description of Artificial Sequence Primer
ggagttggtaatctaactgatgc

SEQ ID NO: 73 - DNA, 21 bp, Artificial Sequence
  Description of Artificial Sequence Primer
ccaacagcaacattatctgaa

SEQ ID NO: 74 - DNA, 20 bp, Artificial Sequence
  Description of Artificial Sequence Primer
gccctcttgtggcataaatc

SEQ ID NO: 75 - DNA, 20 bp, Artificial Sequence
  Description of Artificial Sequence Primer
ctgggagaggaagaaccgta

SEQ ID NO: 76 - DNA, 20 bp, Artificial Sequence
  Description of Artificial Sequence Primer
tacttggggctctcagcttg

SEQ ID NO: 77 - DNA, 21 bp, Artificial Sequence
  Description of Artificial Sequence Primer
ggcactcctgaagtaggaagg

SEQ ID NO: 78 - DNA, 20 bp, Artificial Sequence
  Description of Artificial Sequence Primer
ccctccctgtttttgttgaa

SEQ ID NO: 79 - DNA, 20 bp, Artificial Sequence
  Description of Artificial Sequence Primer
gggcaaagacagtcaggatt

SEQ ID NO: 80 - DNA, 20 bp, Artificial Sequence
  Description of Artificial Sequence Primer
ctgggccatttgtttaactt

SEQ ID NO: 81 - DNA, 20 bp, Artificial Sequence
  Description of Artificial Sequence Primer
tgaaattgtctcccaagatg

SEQ ID NO: 82 - DNA, 20 bp, Artificial Sequence
  Description of Artificial Sequence Primer
tagttaaagacgggcctatt

SEQ ID NO: 83 - DNA, 20 bp, Artificial Sequence
  Description of Artificial Sequence Primer
caagagtagctttggtggag

SEQ ID NO: 84 - DNA, 20 bp, Artificial Sequence
  Description of Artificial Sequence Primer
aagactggagggatagcagt

SEQ ID NO: 85 - DNA, 24 bp, Artificial Sequence
  Description of Artificial Sequence Primer
agatcttaggtcatttcttctttg

SEQ ID NO: 86 - DNA, 23 bp, Artificial Sequence
  Description of Artificial Sequence Primer
tgaactgattttcacattaccac

SEQ ID NO: 87 - DNA, 20 bp, Artificial Sequence
  Description of Artificial Sequence Primer
aattggcagcacttagaacc

SEQ ID NO: 88 - DNA, 20 bp, Artificial Sequence
  Description of Artificial Sequence Primer
gccttcagggttgtcttact

SEQ ID NO: 89 - DNA, 21 bp, Artificial Sequence
  Description of Artificial Sequence Primer
cattgtaatcacacaagcaaa

SEQ ID NO: 90 - DNA, 20 bp, Artificial Sequence
  Description of Artificial Sequence Primer
acagagggtgcagttcaggt

SEQ ID NO: 91 - DNA, 20 bp, Artificial Sequence
  Description of Artificial Sequence Primer
cacaatggttgccaatatgc

SEQ ID NO: 92 - DNA, 20 bp, Artificial Sequence
  Description of Artificial Sequence Primer
aaggtcaagggagagaacct

SEQ ID NO: 93 - DNA, 20 bp, Artificial Sequence
  Description of Artificial Sequence Primer
acctctgctctcagatgctt

SEQ ID NO: 94 - DNA, 20 bp, Artificial Sequence
  Description of Artificial Sequence Primer
catgcacacccactgtactt

SEQ ID NO: 95 - DNA, 21 bp, Artificial Sequence
  Description of Artificial Sequence Primer
ctggaacactactgccagttt

SEQ ID NO: 96 - DNA, 20 bp, Artificial Sequence
  Description of Artificial Sequence Primer
ctttgggattcaataatgct

SEQ ID NO: 97 - DNA, 21 bp, Artificial Sequence
  Description of Artificial Sequence Primer
ccaatctgctggtattaagaa

SEQ ID NO: 98 - DNA, 20 bp, Artificial Sequence
  Description of Artificial Sequence Primer
tgtggaatctgtgggatgtg

SEQ ID NO: 99 - DNA, 20 bp, Artificial Sequence
  Description of Artificial Sequence Primer
tgctaaggaccgcttacgtt

SEQ ID NO: 100 - DNA, 20 bp, Artificial Sequence
  Description of Artificial Sequence Primer
ttttgccagcactgatttta

SEQ ID NO: 101 - DNA, 20 bp, Artificial Sequence
  Description of Artificial Sequence Primer
tttcaagtctgcaattttgg

SEQ ID NO: 102 - DNA, 20 bp, Artificial Sequence
  Description of Artificial Sequence Primer
caactcccatagccctttgc

SEQ ID NO: 103 - DNA, 20 bp, Artificial Sequence
  Description of Artificial Sequence Primer
taatttacccgccagggaca

SEQ ID NO: 104 - DNA, 20 bp, Artificial Sequence
  Description of Artificial Sequence Primer
gaggtagcagcaatccctaa

SEQ ID NO: 105 - DNA, 23 bp, Artificial Sequence
  Description of Artificial Sequence Primer
catggattgatttcagaattttt

SEQ ID NO: 106 - DNA, 21 bp, Homo sapiens
gcctggttactttaaggatgg

SEQ ID NO: 107 - DNA, 20 bp, Homo sapiens
gcctggttacttaaggatgg

SEQ ID NO: 108 - DNA, 21 bp, Homo sapiens
tatatatcacagtaagtgtct

SEQ ID NO: 109 - DNA, 21 bp, Homo sapiens
tatatatcacggtaagagtct

SEQ ID NO: 110 - protein, 36 aa, Homo sapiens
SRYLTDFEPIQCLGRGGFGVVFEAKNKVDDCNYAIK

SEQ ID NO: 111 - protein, 36 aa, Mus musculus
SRYLTDFEPIQCMGRGGFGVVFEAKNKVDDCNYAIK

SEQ ID NO: 112 - protein, 36 aa, Rattus norvegicus; identical to SEQ ID NO: 111

SEQ ID NO: 113 - protein, 36 aa, Saccharomyces cerevisiae
SRYASDFEEIAVLGQGAFGQVVKARNALDSRYYAIK

SEQ ID NO: 114 - protein, 36 aa, Mus musculus
SRYFIEFEELQLLGKGAFGAVIKVQNKLDGCCYAVK

SEQ ID NO: 115 - protein, 36 aa, Caenorhabditis elegans
SRFANEFEVKKVIGHGGFGVVFRAQSITDMNEYAVK

SEQ ID NO: 116 - protein, 36 aa, Drosophila melanogaster
SRLRTEFEVLMYLGKGAFGDVLKVRNILDNREYAIK

SEQ ID NO: 117 - protein, 37 aa, Schizosaccharomyces pombe
SRYIEDFEEYSLLGRGGFGSVYHVRNKIDGAEYAMKK

SEQ ID NO: 118 - protein, 36 aa, Schizosaccharomyces pombe
SRYASDFEELELLGKGGYGSVYKARNKFDGVEYALK

SEQ ID NO: 119 - protein, 36 aa, Homo sapiens
SRYLNEFEELVILGKGGYGRVYKVRNKLDGQYYAIK

SEQ ID NO: 120 - protein, 36 aa, Mus musculus
SRYLNEFEELAILGKGGYGRVYKVRNKLDGQHYAIK

SEQ ID NO: 121 - protein, 36 aa, Rattus norvegicus; identical to SEQ ID NO: 120

SEQ ID NO: 122 - protein, 36 aa, Oryctolagus cuniculus
SRYLNEFEELSILGKGGYGRVYKVRNKLDGQYYAIK

SEQ ID NO: 123 - protein, 36 aa, Homo sapiens
SRYTTEFHELEKIGSGEFGSVFKCVKRLDGCIYAIK

SEQ ID NO: 124 - protein, 36 aa, Mus musculus; identical to SEQ ID NO: 123

SEQ ID NO: 125 - protein, 36 aa, Rattus norvegicus; identical to SEQ ID NO: 123

SEQ ID NO: 126 - protein, 36 aa, Zea mays
SRYRTDFHEIEKIGYGNFSVVFKVLNRIDGCLYAVK

SEQ ID NO: 127 - protein, 36 aa, Arabidopsis thaliana
SRYLTDFHEIRQIGAGHFSRVFKVLKRMDGCLYAVK

SEQ ID NO: 128 - protein, 36 aa, Drosophila melanogaster
SRFKREFMQVNVIGVGEFGVVFQCVNRLDGCIYAIK

SEQ ID NO: 129 - protein, 36 aa, Xenopus laevis
SRYKTEFLEIEKIGAGEFGSVFKCVKRLDGCFYAIK

SEQ ID NO: 130 - protein, 35 aa, Homo sapiens
RFGMDFKEIELIGSGGFGQVFKAKHRIDGKTYVIK

SEQ ID NO: 131 - protein, 36 aa, Mus musculus
ARFNSDFEDIEEIGLGGFGQVFKAKHRIDGKRYAIK

SEQ ID NO: 132 - protein, 35 aa, Rattus norvegicus
RFSKDFEDIEEIGSGGFGQVFKAKHRIDGKTYAIK

(19) United States
(12) Patent Application Publication
(10) Pub. No.: US 2004/0180338 A1
(43) Pub. Date: Sep. 16, 2004

(54) Mutated eukariotic transalation initiation factor 2 alpha kinase3, eif2ak3, in patients with neonatal insuluin-dependant diabetes and multiple epiphyseal dyslapsia (wolcott-rallison syndrome)

(76) Inventors: Marc Delepine, Paris (FR); Marc Nicolino, Lyon (FR); Cecile Julier, Paris (FR)

Correspondence Address: FOLEY AND LARDNER, SUITE 500, 3000 K STREET NW, WASHINGTON, DC, 20007, US

(21) Appl. No.: 10/296,450

(22) PCT Filed: May 23, 2001
(86) PCT No.: PCT/IB01/01153

(30) Foreign Application Priority Data
May 23, 2000 (EP) 00401436.1
Oct. 2, 2000 (EP) 00402707.4

(51) Int. Cl. C12Q 1/68; C07H 21/04; C12N 9/12

(52) U.S. Cl. 435/6; 435/69.1; 435/194; 435/320.1; 435/325; 536/23.2

(57) ABSTRACT

The present invention is directed to isolated variant nucleic sequence of genomic sequence encoding the translation initiation factor 2 alpha kinase 3 (EIF2AK3) capable of inducing the Wolcott-Rallison syndrome (WRS) or affecting the risk of developing diabetes and/or other pathology related to WRS, and to the polypeptide encoded by these sequences. The invention also relates to vectors or transformned cells containing these sequences. The present invention further concerns method and kit for determining in a subject the risk of developing diabetes and/or other pathology related to WRS and method for selecting compound which can be used as medicament for the prevention and/or treatment of these pathologies.

The present invention is directed to isolated variant nucleic sequence of genomic sequence encoding the translation initiation factor 2 alpha kinase 3 (EIF2AK3) capable of inducing the Wolcott-Rallison syndrome (WRS) or affecting the risk of developing diabetes and/or other pathology related to WRS, and to the polypeptide encoded by these sequences. The invention also relates to vectors or transformed cells containing these sequences. The present invention further concerns method and kit for determining in a subject the risk of developing diabetes and/or other pathology related to WRS and method for selecting compound which can be used as medicament for the prevention and/or treatment of these pathologies.

Wolcott-Rallison syndrome (WRS) is characterized by insulin-dependent diabetes with neonatal onset, or occurrence in early infancy, associated with multiple epiphyseal dysplasia and osteoporosis that appear at a later age. Other conditions that may be associated with WRS include hepatic and renal dysfunction, mental retardation, skin abnormality (ectodermal dysplasia) and teeth discoloration and cardiovascular abnormalities (Wolcott, C. D., et al., J. Pediatr., 80, 292-297, 1972; Goumy, P., et al., Arch Fr Pediatr., 37, 323-328, 1980 ; Stoss, H., et al., Eur J Pediatr., 138, 120-129, 1982; Al-Gazali, L. I., et al., Clinical Dysmorphology, 4, 227-233, 1995 ; Thornton, C. M., et al., Pediatr. Pathol. Lab. Med., 17, 487-96, 1997). Although insulin replacement therapy is required from the onset of diabetes, the etiology does not appear to be autoimmune since anti-islet cell and other diabetes related auto-antibodies are absent in WRS patients. Autopsy exploration of the pancreas reveals major decrease in pancreatic β cells (Nicolino, P. M., et al., Hormone Research, 50, A215, 1998).

The syndrome was first described in 1972 by Wolcott and Rallison in a single family with three affected siblings (Wolcott, C. D., et al.). Subsequently, approximately 10 cases have been reported in the literature. Despite the rarity of the syndrome, the available data argue strongly that the syndrome is inherited as an autosomal recessive trait: the disease is absent in the parents of WRS patients; its incidence appears to be independent of sex; the disease recurs in siblings; and it is found predominately in children born of consanguineous marriages. The incidence of WRS may be under-estimated because patients may die at a very young age from diabetes or other pathological manifestations before the full syndrome is apparent, and then be misdiagnosed with neonatal or early onset insulin-dependent diabetes mellitus (IDDM). Rare Mendelian subtypes of multifactorial disorders may provide insights to identify novel disease pathways, and for investigation of susceptibility in other more common forms of disease. For example, milder variants of the gene responsible for WRS could also be involved in the susceptibility to the common form of IDDM as well as to the other associated features, thus lending a particular interest to study of this syndrome. The investigation of other rare forms of diabetes such as Wolfram syndrome ⁷, maturity onset diabetes of the young (MODY) (for recent reviews, see Inoue, H. et al., Nat. Genet., 20, 143-8, 1998 ; Hattersley, A. T., Diabet. Med. 15, 15-24, 1998 ; Winter, W. E., et al., Endocrinol Metab. Clin. North. Am., 28, 765-85, 1999 ; Froguel, P., et al., Trends in Endocrinology and Metabolism, 10, 142-146, 1999), and metabolic syndrome with insulin resistance associated with diabetes and hypertension (Barroso, I., et al., Nature, 402, 880-3 1999) have all revealed novel disease mechanisms of biological interest.

The pathogenic pathways involved in WRS are unknown, but for reasons given above the disorder is most likely to be due to a single gene responsible for the pleiotropic features. Because of the complete absence of pancreatic β-cells, with no evidence of an autoimmune process, it has been proposed that this gene may be involved in the development of the endocrine pancreas. Recently, a detailed investigation of the paired-box transcription factor PAX4 gene, which has been previously shown to be important in the differentiation of pancreatic β cells (Sosa-Pineda, et al., Nature, 386, 399-402, 1997), excluded it as being the gene responsible (Bonthron, D. T., et al., J. Med. Genet., 35, 288-92, 1998). A single case report of the presence of WRS syndrome in a patient with a mosaic deletion of chromosome 15q11-12 raised the possibility that the gene is located in this chromosome region (Stewart, F. J. et al,. Clin. Genet. 49, 152-5, 1996). However, no confirmation of this potential localization has been reported.

The molecular mechanisms responsible for diabetes in man are complex and involve genetic and environmental factors. It is important to define the genetic mechanisms which are involved in diabetes so as to be able anticipate the risk of developing these pathologies and/or to develop better targeted medicaments.

The inventors have examined WRS in two consanguineous families with different ethnic origins, one of Tunisian descent and the other of Pakistanese descent. A genome-wide linkage study was undertaken in the first family, which has three affected and one unaffected offspring, leading to the identification of a probable localization of a gene involved in the disease in a 17 cM region on chromosome 2. The localization was confirmed in the second family, and the combined data allowed us to map the gene to an interval of 2-3 cM defined by recombination events at the distal and proximal boundaries. Amongst the genes mapped to the interval, we identified the eukaryotic translation initiation factor 2 alpha kinase 3 (EIF2AK3), also known as pancreatic eukaryotic initiation factor 2α-subunit kinase (PEK) as a major candidate gene for WRS because of its high level of expression in the pancreas islet. This gene is also highly expressed in the placenta, where normal expression from the mother may explain the healthy status of WRS patients at birth, followed by rapid onset of diabetes.

The present invention therefore relates to an isolated variant nucleic sequence of a mammal genomic sequence of the gene encoding the translation initiation factor 2 alpha kinase 3 (EIF2AK3), said EIF2AK3 protein having the sequence SEQ ID No. 2, characterized in that the presence of said variant sequence in a mammal is capable of inducing the Wolcott-Rallison syndrome (WRS) or affects the risk of onset or progression of diabetes and/or pathology related to WRS.

It should be understood that the invention does not relate to nucleic sequences or polypeptides in a natural form, that is to say that they are not taken in their natural environment but that it may have been possible for them to be obtained by purification from natural sources, or alternatively obtained by genetic recombination, or alternatively by chemical synthesis.

Nucleic sequence or nucleic acid is understood to mean an isolated natural, or a synthetic, DNA and/or RNA fragment comprising, or otherwise, non natural nucleotides, designating a precise succession of nucleotides, modified or otherwise, allowing a fragment, a segment or a region of a nucleic acid to be defined.

Variant nucleic sequence (or protein, polypeptide or peptide variant) will be understood to mean all the alternative nucleic sequence (or alternative polypeptide) which may naturally exist, in particular in human beings, and which correspond in particular to deletions, substitutions and/or additions of nucleotides (or amino acid residues). In the present case, the variant nucleic sequence (or variant polypeptide) will be in particular partly associated with the risk of onset or progression of diabetes and/or pathology related to WRS. Thus, They may be associated with predisposition, or resistance or protection against the onset and/or progression of diabetes and/or pathology related to WRS, preferably associated with predisposition to the onset and/or progression of diabetes and/or pathology related to WRS (increase risk).

In the present description, “diabetes and/or pathology related to WRS” will be understood to mean type 1 diabetes, type 2 diabetes and others forms of diabetes and/or other pathologies which have been described in patients affected by the WRS, in particular bone disorders, such as osteoporosis and arthritis, hepatic dysfinction, nephropathies or other renal dysfunction, mental retardation, skin abnormality, teeth discoloration and cardiovascular abnormalities.

Among the bone disorders related to the WRS, achondrogenesis, epiphyseal dysplasia, achondroplasia, hypochondroplasia, can be also particularly cited.

Normal nucleic sequence or normal variant nucleic sequence (or normal variant polypeptide) will be understood to mean a nucleic sequence or a variant nucleic (or normal variant polypeptide) which does not affect the risk of onset and/or progression of diabetes and/or pathology related to VWRS in a mammal, particularly in human being.

“Affect the risk of onset and/or progression of diabetes and/or pathology related to WRS” will be understood to mean increase (predisposition) or decrease (resistance or protection) of the probability for a subject to develop (onset or progression) diabetes and/or pathology related to WRS in a mammal, particularly in human being.

Allele or allelic variant will be understood to mean the natural alternative sequences corresponding to polymorphisms present in human beings and, in particular, to polymorphisms which can affect the risk of having the WRS or the risk of onset and/or progression of diabetes and/or pathology related to WRS, in particular at the type 1 or the type 2 diabetes level, or at the other forms of diabetes level, preferably at the type 1 diabetes level.

Alternative nucleic sequences are understood to mean preferably the nucleic sequences comprising at least one point variation compared with the normal sequence and preferably at most 10%, preferably 5%, 2.5%, 2%, 1.5% and 1% of point variations compared with the normal sequence.

Preferably, the present invention relates to alternative nucleic sequences in which the point variations are not silent, that is to say that they lead to a modification of the amino acid encoded in relation to the normal sequence. Still more preferably, these point variations affect amino acids which are located in the catalytic site of the normal protein.

Acid nucleic fragment (or polypeptide fragment) is understood to mean an acid nucleic fragment (or polypeptide or a peptide encoded by) comprising a minimum of 12 nucleotides or bases, preferably 15, 20, 25, 30, 40 or 50 bases. These fragments may comprise in particular a point variation, compared with a nucleic sequence which does not affect the risk of developing diabetes and/or pathology related to WRS in a mammal (normal variant nucleic acid), particularly in human being.

In a preferred embodiment, this isolated variant nucleic sequence according to the invention, is characterized in that said diabetes and/or pathology related related to WRS are selected from the group consisting of type 1 diabetes, type 2 diabetes, the others forms of diabetes, osteoporosis, arthritis, hepatic dysfunction, nephropathies and other renal dysfinction and mental retardation.

In another preferred embodiment, this isolated variant nucleic sequence is characterized in that said diabetes and/or pathology related to WRS is selected from the group consisting of type 1 diabetes, type 2 diabetes, and other forms of diabetes.

In a more preferred embodiment, this isolated variant nucleic sequence is characterized in that said diabetes and/or pathology related to WRS is selected from the group consisting of type 1 diabetes and type 2 diabetes.

In a particular more preferred embodiment, this isolated variant nucleic sequence is characterized in that said diabetes and/or pathology related to WRS is type 1 diabetes.

The subject of the invention is also an isolated variant nucleic sequence according to the invention, characterized in that said diabetes and/or pathology related to WRS is linked to a loss, particularly a major decrease, of pancreatic β-cells or integrity thereof

The invention relates to isolated variant nucleic sequence according to the invention, characterized in that said diabetes and/or pathology related to WRS results from the alteration of the control which is exerted by EIF2AK3 on a specific protein from the pancreas and/or from the chondrocytes, said control, if normally exerted, insuring the adequate development and function of these organs.

Also preferred among the isolated variant nucleic sequence according to the invention are the isolated variant nucleic sequence comprising or consisting of a nucleic acid sequence selected from the group consisting of the sequences SEQ ID No. 3 to No. 15 or fragment thereof, provided said isolated variant nucleic sequence is not the sequence SEQ ID No. 1.

In a preferred embodiment, the present invention relates to an isolated variant nucleic sequence according to the invention, characterized in that the protein EIF2AK3 encoded by said variant sequence presents at least one variation compared to the sequence SEQ ID No. 2 of EIF2AK3.

In an also preferred embodiment, the present invention relates to isolated variant nucleic sequence according to the invention, characterized in that the protein EIF2AK3 encoded by said variant sequence presents a premature termination or at least one variation in the catalytic domain aa 576-aa 1115 of the protein EIF2AK3 having the sequence SEQ ID No. 2.

The isolated variant nucleic sequence according to he invention, characterized in that said sequence comprises an insertion of a T at position 1103 or a G to A transition at position 1832 in the sequence SEQ ID No. 1, also forms part of the present invention.

In a further preferred embodiment, the present invention concerns an isolated variant nucleic sequence according to the invention, characterized in that said sequence comprises at least one of the nucleic sequence polymorphisms which are defined in Tables 4 A and B, and in Table 5, column “cDNA position” and/or “genomic DNA position”.

In a more preferred embodiment, the present invention concerns an isolated variant nucleic sequence according to the invention, characterized in that said sequence is chosen from a human nucleic sequence.

Complementary sequence of the variant nucleic sequence according to the present invention is also comprised in the present invention.

In another object, the present invention is directed to polypeptide encoded by the isolated variant nucleic sequence according to the invention, characterized in that its amino acids sequence presents at least one point variation compared to the sequence SEQ ID No. 2 of EIF2AK3.

In a preferred embodiment, the present invention concerns a polypeptide according to the invention, characterized in that it comprises at least one of the amino acid variations as listed in the column “amino acid” in Tables 4A and 5.

Isolated nucleic acid sequence, characterized in that it encodes the polypeptide according to the invention, also forms part of the present invention.

The invention further comprises isolated nucleic acid sequence, characterized in that it is selected from the group consisting of:

a) a fragment of nucleic sequence according to the invention and comprising at least 12, 15, 20, 25, 30, 40 or 50 bases;

b) a nucleic sequences capable of hybridizing specifically with the nucleic sequence as defined in a)and comprising at least 12, 15, 20, 25, 30, 40 or 50 bases.

“Nucleic sequences capable of hybridizing specifically” with a reference nucleic sequence will be understood to mean a nucleic sequence capable of hybridizing with said reference sequence under stringent conditions, conditions which are well known by a skilled person and which can be found for example in Sambrook et al. (Molecular Cloning, A Laboratory Manual, Sec. Edition, Cold Spring Harbor Laboratory Press, pages 11.50 and 11.51, 1989).

Preferably, the nucleic sequences capable of hybridizing specifically with the reference sequence have at least 80%, 85%, 90%, 95% or 99% identity degree after optimal alignment with the complementary sequence of the reference sequence.

The term “identity degree” refers in the present description to degree or percentage of identity between two sequences after optimal alignment as defined below in the present application.

Two amino-acids or nucleotidic sequences are said to be “identical” if the sequence of amino-acids or nucleotidic residues, in the two sequences is the same when aligned for maximum correspondence. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2: 482 (1981), by the homology alignment algorithm of Neddleman and Wunsch, J. Mol. Biol. 48:443 (1970), by the search for similarity method of Pearson and Lipman, Proc. Natl. Acad. Sci. (U.S.A.) 85:2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection.

“Identity degree” is determined by comparing two optimally aligned sequences over a comparison window, where the portion of the peptide or polynucleotide sequence in the comparison window may comprise additions or deletions (i.e., gaps) as compared lo to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

The definition of sequence identity given above is the definition that would use one of skill in the art. The definition by itself does not need the help of any algorithm, said algorithms being helpful only to achieve the optimal alignments of sequences, rather than the calculation of sequence identity.

From the definition given above, it follows that there is a well defined and only one value for the sequence identity between two compared sequences which value corresponds to the value obtained for the best or optimal alignment.

In the BLAST N or BLAST P “BLAST 2 sequence” Tatusova et al., “Blast 2 sequences—a new tool for comparing protein and nucleotide sequences”, FEMS 25 Microbiol. Lett. 174:247-250) software which is available in the web site http://www.ncbi.nlm.nih.gov/gorf/b12.html, and habitually used by the inventors and in general by the skilled man for comparing and determining the identity between two sequences. The “open gap penalty” and <<extension gap penalty >> parameters which depends on the substitution matrix selected regarding the nature and the length of the 30 sequence to be compared is directly selected by the software (i.e. “5” and “2” respectively for substitution matrix BLOSUM-62). The identity percentage between the two sequences to be compared is directly calculated by the software.

According to the invention, the fragments of nucleic sequences may be used as probe or as primer in methods of detection, identification or amplification of nucleic sequence. These fragments have a minimum size of 10 bases and fragments of at least 20 bases, preferably 25 and 30 bases, will be preferred.

The nucleic sequences which can be used as primer or probe, characterized in that their nucleic sequence is a sequence of the invention, also form part of the invention.

The present invention relates to all the primers which may be deduced from the nucleotide sequences of the invention and which may make it possible to detect the said nucleotide sequences of the invention, in particular the alternative sequences, using in particular a method of amplification such as the PCR method, or a related method.

The present invention relates to all the probes which may be deduced from the nucleotide sequences of the invention, in particular sequences capable of hybridizing with them, and which may make it possible to detect the said nucleotide sequences of the invention, in particular to discriminate between the normal sequences and the alternative sequences.

All the probes and primers according to the invention may be labeled by methods well known to persons skilled in the art, in order to obtain a detectable and/or quantifiable signal.

The present invention relates, of course, to both the DNA and RNA sequences, as well as the sequences which hybridize with them.

So, the present invention concerns isolated nucleic acid sequence according to the invention as a primer or a probe.

In a preferred embodiment, the invention relates to isolated nucleic acid sequence according to the invention, characterized in that it is selected from the group consisting of sequences SEQ ID No. 16 to SEQ ID No. 105.

In another aspect, the invention comprises nucleic acid sequence which can be used as sense or anti-sense oligonucleotide, characterized in that its sequence is chosen from the sequences according to the invention.

Among the nucleic acid fragments of interest, there should thus be mentioned, in particular the anti-sense oligonucleotides, that is to say whose structure ensures, by hybridization with the target sequence, inhibition of the expression of the corresponding product. There should also be mentioned the sense oligonucleotides which, by interaction with the proteins involved in the regulation of the expression of the corresponding product, will induce either inhibition, or activation of this expression.

Also included in the invention are the nucleic acid sequences of a promoter and/or regulator of the EIF2AK3 gene, or one of their allelic variants, or one of their fragments, characterized in that they are capable of being obtained from the nucleic sequence of the invention.

The sequences carrying variations which may be involved in the promoter and/or regulatory sequences of the EIF2AK3 gene and which may have effects on the expression of the corresponding protein, in particular on their level of expression, also form part of the preceding sequences according to the invention.

It is indeed possible, using the genomic sequences of the EIF2AK3 gene, to identify the polymorphisms present in their promoters and/or regulators, in a population of human subjects, and more specifically of patients suffering or at risk from the pathologies mentioned above.

Among these sequences SEQ ID No. 3 to No. 15, the sequence SEQ ID No. 3, which corresponds to a non coding domain, comprises the sequence encoded the promoter and/or regulator sequence of the EIF2AK3 gene. Said sequence or active fragments thereof are of particular interest for identifying their promoters and/or regulators, and polymorphism thereof. Said promoters and/or regulators can also be used for targeting the expression of EIF2AK3 polypeptide, or variants thereof, or heterologous protein in cells where the EIF2AK3 gene is naturally expressed, such as pancreatic β-cells.

Among the nucleic fragments which may be of interest, in particular for diagnosis, there should be mentioned, for example, the genomic intron sequences of the EIF2AK3 gene, such as in particular the joining sequences between the introns and the exons of normal alternative sequence(s) or alternative sequence(s) which affects the risk of having WRS or the risk of having (onset and/or progression of) diabetes and/or pathology related to WRS.

The invention also comprises the cloning and/or expression vectors containing a nucleic acid sequence according to the invention.

The vectors according to the invention, characterized in that they comprise the elements allowing the expression and/or the secretion of the said sequences in a host cell, also form part of the invention.

The said vectors will preferably comprise a promoter, signals for initiation and termination of translation, as well as appropriate regions for regulation of transcription. They must be able to be stably maintained in the cell and may optionally possess particular signals specifying the secretion of the translated protein.

These different control signals are chosen according to the cellular host used. To this end, the nucleic acid sequences according to the invention may be inserted into autonomously replicating vectors inside the chosen host, or integrative vectors of the chosen host.

Among the autonomously replicating systems, there will be preferably used according to the host cell, systems of the plasmid or viral type, it being possible for the viral vectors to be in particular adenoviruses, retroviruses, pox viruses or herpesviruses Persons skilled in the art know the technologies which can be used for each of these systems.

When the integration of the sequence into the chromosomes of the host cell is desired, it will be possible to use, for example, systems of the plasmid or viral type; such viruses will be, for example, retroviruses.

Such vectors will be prepared according to the methods commonly used by persons skilled in the art, and the clones resulting therefrom may be introduced into an appropriate host by standard methods such as, for example, lipofection, electroporation or heat shock.

The invention comprises, in addition, the host cells, in particular eukaryotic and prokaryotic cells, transformed by the vectors according to the invention, as well as the mammals, except man, comprising one of the said transformed cells according to the invention.

Among the cells which can be used for these purposes, there may of course be mentioned bacterial cells but also yeast cells, animal cells, in particular mammalian cell cultures, and in particular Chinese hamster ovary cells (CHO), but also insect cells in which it is possible to use methods using baculoviruses, for example Sf9 cells.

Among the mammals according to the invention, there will be preferred animals such as mice, rats or rabbits, expressing a polypeptide according to the invention, the phenotype corresponding to the normal or variant EIF2AK3, in particular alternative of human origin.

Among the animal models more particularly of interest here, there are in particular:

transgenic animals exhibiting a deficiency in the expression of EIF2AK3 gene. They are obtained by homologous recombination on embryonic stem cells, transfer of these stem cells to embryos, selection of the chimeras affected at the level of the reproductive lines, and growth of the said chimeras;

transgenic mice overexpressing one or more of a EIF2AK3 gene allelic variant of murine and/or human origin. The mice are obtained by transfection of multiple copies of said EIF2AK3 gene allelic variant under the control of a strong promoter of an ubiquitous nature, or selective for a type of tissue, preferably the pancreatic organ;

transgenic animals made deficient in EIF2AK3 gene part by inactivation with the aid of the LOXP/CRE recombinase system or any other system for inactivating the expression of a gene at a precise age of the animal;

animals (preferably rats, rabbits, mice) over-expressing EIF2AK3 gene, after viral transcription or gene therapy.

The invention also relates to the use of a nucleic acid sequence according to the invention for the production of recombinant or synthetic polypeptides.

The polypeptides obtained by chemical synthesis and which are capable of comprising non natural amino acids corresponding to the said recombinant polypeptides are also included in the invention.

These polypeptides may be produced from the nucleic acid sequences defined above, according to techniques for the production of recombinant polypeptides known to persons skilled in the art. In this case, the nucleic acid sequence used is placed under the control of signals allowing its expression in a cellular host.

An effective system of production of a recombinant polypeptide requires having a vector and a host cell according to the invention.

These cells may be obtained by introducing into the host cells a nucleotide sequence inserted into a vector as defined above, and then culturing the said cells under conditions allowing the replication and/or expression of the transfected nucleotide sequence.

These cells can be used in a method for the production of a recombinant polypeptide according to the invention and can also serve as a model for analysis and screening.

The method for the production of a polypeptide of the invention in recombinant form is itself included in the present invention, and is characterized in that the transformed. cells are cultured under conditions allowing the expression of a recombinant polypeptide of nucleic acid sequence according to the invention, and in that the said recombinant polypeptide is recovered.

The mono- or polyclonal antibodies or fragments thereof, chimeric or immunoconjugated antibodies, characterized in that they are capable of specifically recognizing a polypeptide according to the invention, also form part of the invention.

Specific polyclonal antibodies may be obtained from a serum of an animal immunized against the variant polypeptides of the invention, particularly against variant polypeptides of the invention which are alternative compared with the normal amino-acids sequence, said variant polypeptides can be produced by genetic recombination or by peptide synthesis, according to the customary procedures, from a nucleic acid sequence according to the invention.

There may be noted in particular the advantage of antibodies specifically recognizing certain variant polypeptides, or fragments thereof, which are of particular interest, according to the invention.

The specific monoclonal antibodies may be obtained according to the conventional hybridoma culture method.

The antibodies according to the invention are, for example, chimeric antibodies, humanized antibodies, Fab or F(ab′)2 fragments. They may also be in the form of immunoconjugates or of labeled antibodies so as to obtain a detectable and/or quantifiable signal.

The invention also relates to methods for the detection and/or purification of a variant polypeptide according to the invention, characterized in that they use an antibody according to the invention.

Moreover, the antibodies of the invention, in particular the monoclonal antibodies, may also be used for the detection of these polypeptides in a biological sample.

They thus constitute a means for the immunocytochemical or immuno-histochemical analysis of the expression of said variant EIF2AK3 polypeptide on specific tissue sections, for example by immunofluorescence, gold labeling, enzymatic immunoconjugates.

They make it possible in particular to detect abnormal expression of these polypeptides in the biological tissues or samples, which makes them useful for the detection of abnormal expression of EIF2AK3 polypeptide or for monitoring the progress of a method of prevention or treatment of diabetes and/or pathology related to WRS.

Also forming part of the invention are the methods for the determination of an allelic variability, for example the presence of a EIF2AK3 gene variation, such as the presence of a deletion, substitution and/or addition of nucleotide(s), a loss of heterozygosity or a genetic abnormality, characterized in that they use a nucleic acid sequence according to the invention.

The determination of an allelic variability, a loss of heterozygosity or a genetic abnormality in a tested subject according to the method of the invention, will permit for example the identification of a subject who exhibits an alternative sequence of the EIF2AK3 gene, present on one or each allele, associated with a predisposition to or with a resistance or protection against the risk of onset or progression of diabetes and/or pathology related to WRS, by comparing the sequence of the tested subject with the sequence(s) of the EIF2AK3 gene of subject(s) who does not present a decrease or increase risk of onset or progression of diabetes and/or pathology related to WRS, or by determining if the tested subject presents or does not present an allelic identity with subject(s) known to have WRS or to be at decreased or increased risk of having diabetes and/or pathology related to WRS.

These diagnostic methods relate to, for example, the methods for the antenatal or postnatal diagnosis of risk of having VWRS or for diagnosis of predisposition, or resistance or protection, to diabetes and/or pathology related to WRS, linked for example with abnormalities in the expression of the EIF2AF3 protein, by determining, in a biological sample from the patient, the presence of variation in one of the sequences described above. The nucleic acid sequences analyzed may be either the genomic DNA, the cDNA or the MRNA.

The nucleic acid tools based on the present invention can also allow a positive and differential diagnosis in a patient taken in isolation. They will be preferably used for a presymptomatic diagnosis in an at risk subject, in particular with a familial history. It is also possible to envisage an antenatal or in a newborn diagnosis.

In addition, the detection of a specific variation may allow an evolutive diagnosis, in particular as regards the intensity of the pathology or the probable period of its appearance.

The methods allowing the detection of variation in a gene compared with the natural gene are, of course, highly numerous. They can essentially be divided into two large categories. The first type of method is that in which the presence of a variation is detected by comparing the alternative sequence with the corresponding normal sequence(s), and the second type is that in which the presence of the variation is detected indirectly, for example by evidence of the mismatches due to the presence of the variation.

Among the methods for the determination of an allelic variability, a loss of heterozygocity or a genetic abnormality, such as a variation, the methods comprising at least one stage for the so-called PCR (polymerase chain reaction) or PCR-like amplification of the target sequence according to the invention likely to exhibit an abnormality with the aid of a pair of primers of nucleotide sequences according to the invention are preferred. The amplified products may be treated with the aid of an appropriate restriction enzyme before carrying out the detection or assay of the targeted product.

PCR-like will be understood to mean all methods using direct or indirect reproductions of nucleic acid sequences, or alternatively in which the labeling systems have been amplified, these techniques are of course known, in general they involve the amplification of DNA by a polymerase; when the original sample is an RNA, it is advisable to carry out a reverse transcription beforehand. There are currently a great number of methods allowing this amplification, for example the so-called NASBA “Nucleic Acid Sequence Based Amplification”, TAS “Transcription based Amplification System”, LCR “Ligase Chain Reaction”, “Endo Run Amplification” (ERA), “Cycling Probe Reaction” (CPR), and SDA “Strand Displacement Amplification”, methods well known to persons skilled in the art.

Variation in the EIF2AK3 gene may be responsible for various modifications of their products, which modifications can be used for a diagnostic approach. Indeed, modifications of antigenicity can allow the development of specific antibodies. The discrimination between the different products can be achieved by these methods. All these modifications may be used in a diagnostic approach by virtue of several well known methods based on the use of mono- or polyclonal antibodies capable of specifically recognizing the EIF2AK3 polypeptide variants, such as for example using RIA or ELISA.

Thus, the present invention is directed to a method for the diagnosis of diabetes and/or pathology related to WRS or correlated with an abnormal expression of a polypeptide having the sequence SEQ ID No. 2, characterized in that one or more antibodies according to the invention is(are) brought into contact with the biological material to be tested, under conditions allowing the possible formation of specific immunological complexes between the said polypeptide and the said antibody or antibodies, and in that the immunological complexes possibly formed are detected.

The present invention is further directed to a method for determining if a subject is at decrease or increased risk of having diabetes and/or pathology related to WRS comprising the steps of:

a) collecting a biological sample containing genomic DNA or RNA from the subject;

b) determining on at least one gene allele or RNA encoding the protein EIF2AK3, the sequence, or length thereof, of a fragment of said DNA or RNA susceptible of containing a polymorphism associated to a decrease or increased risk of having diabetes and/or pathology related to WRS, fragment which can be amplified by polymerase chain reaction with a set of primers according to the invention:

c) observing whether or not the subject is at decrease or increased risk of having diabetes and/or pathology related to WRS by observing if the sequence of said fragment of DNA or RNA contains a polymorphism associated to a decrease or increased risk of having diabetes and/or pathology related to WRS, the presence of said polymorphism indicates said subject is at decrease or increased risk of having diabetes and/or pathology related to WRS.

In a preferred embodiment, said method according to the present invention is directed to a method for determining if a subject is at increased risk of having diabetes and/or pathology related to WRS.

The present invention is further directed to an in vitro method (preferably antenatal or in a newborn) for determining if a subject, whose one member of his family is affected by the WRS, is at risk of having WRS comprising the steps of:

a) collecting a biological sample containing genomic DNA or RNA from the subject;

b) determining on the sequence of both alleles of the EIF2AK3 gene, the sequence, or length thereof, of a fragment of said DNA or RNA susceptible of containing a polymorphism associated to the risk of having WRS, fragment which can be amplified by polymerase chain reaction with a set of primers according to the invention;

c) observing whether or not the subject is at risk of having WRS by observing if for both alleles, the sequence of said fragment of DNA or RNA carry a mutation associated to a risk of having WVRS, the presence of said polymorphism indicates said subject is at risk of having WRS.

The present invention is further directed to an in vitro method according to the invention for the diagnosis (preferably antenatal or in a newborn) of the risk of having the WRS, characterized in that said polymorphism associated to the risk of having WRS in step b) is the presence of the mutation corresponding to an insertion of a T at position 1103 or a G to A transition at position 1832 in the sequence SEQ ID No. 1, the presence of said mutation on each of the EIF2AK3 gene allele of said subject indicates said subject is at risk of having WRS.

The present invention is further directed to an in vitro method (preferably antenatal or in a newborn) for determining if a subject, whose one family's member is affected by the WRS, is at risk of having WRS comprising the steps of:

a) collecting a biological sample containing genomic DNA or RNA from the family's member affected by the WRS and from said subject;

b) determining if the family's member affected by the WRS and said subject present an allelic identity by comparing polymorphic markers (microsatellite markers or single nucleotide polymorphisms (SNPs)) which are positioned close to or included in the EIF2AK3 gene, the genotype identity between the family's member affected by the WRS and said subject indicates said subject is at risk of having WRS.

The present invention is further directed to an in vitro method (preferably antenatal or in a newborn) for determining if a subject, whose one family's member is affected by the WRS, is at risk of having WRS comprising the steps of:

a) collecting a biological sample containing genomic DNA or RNA from the family's member affected by the WRS and from said subject;

b) determining on the both EIF2AK3 gene alleles of said family's member, the sequence of a fragment of DNA or RNA susceptible of containing a polymorphism associated to the risk of having WRS, fragment which can be amplified by polymerase chain reaction with a set of primers according to the invention;

c) determining if the mutation of the sequence of said fragments responsible of the WRS affection identified in step b) is present on the same fragment of both the EIF2AK3 gene alleles of said subject, fragment which can be amplified by polymerase chain reaction with a set of primers according to the invention;

d) observing whether or not the subject is at risk of having WRS by observing if the sequence of said fragment on the both EIF2AK3 gene alleles of the subject contains the same mutation as identified in step b), the presence of said mutation on the both alleles indicates said subject is at risk of having WRS.

The present invention is further directed to a method according to the invention, wherein the sequence, or length thereof, of a fragment of DNA or RNA susceptible of containing said polymorphism is obtained in step b) by determining the size of and/or sequencing the amplified products obtained after polymerase chain reaction, eventually after a step of reverse transcription.

The present invention is further directed to a method according to the invention, characterized in that said method further comprises a second method for assaying a biological sample from said subject for levels of at least an additional marker associated with the decreased or increased risk of having diabetes and/or pathology related to WRS, the presence of a significantly level of said at least one marker allowing to confirm if said subject is at decreased or increased risk of having diabetes and/or pathology related to WRS.

In a preferred embodiment, said additional marker associated is an additional marker associated with the increased risk of having diabetes and/or pathology related to WRS.

In another object, the present invention comprises a kit for in vitro determining (preferably antenatal or in a newborn) if a subject is at risk of having WRS, comprising at least one pair of primers capable of amplifing a fragment of genomic DNA containing a polymorphic marker (microsatellite markers or single nucleotide polymorphisms (SNPs)) which is positioned close to or included in the EIF2AK3 gene, and/or at least one probe capable of detecting said polymorphic marker, preferably said at least one pair of primers or probe being chosen among the primers and probes according to the invention.

The present invention further concerns a kit for in vitro determining (preferably antenatal or in a newborn) if a subject is at risk of having WRS, comprising at least one pair of primers capable of amplifying a fragment of EIF2AK3 genomic DNA susceptible to contain an insertion of a T at position 1103 or a G to A transition at position 1832 in the sequence SEQ ID No. 1, said primers being chosen among the primers according to the invention.

The present invention further concerns a kit for determining if a subject is at decreased or increased risk of having diabetes and/or pathology related to WRS, comprising at least one pair of primers capable of amplifying a fragment of genomic DNA or RNA encoding the protein EIF2AK3 and susceptible of containing a polymorphism associated with a decreased or increased risk of having diabetes and/or pathology related to WRS, said primers being chosen among the primers according to the invention.

Preferably said associated polymorphism is a polymorphism associated with an increased risk of having diabetes and/or pathology related to WRS.

The present invention further concerns a kit for according to the invention characterized in that said kit furer comprises means for assaying a biological sample from said subject for levels of at least an additional marker associated with the decreased or increased risk of having diabetes and/or pathology related to WRS, preferably said additional marker associated is an additional marker associated with the increased risk of having diabetes and/or pathology related to WRS.

In a preferred embodiment, said methods or kits as described above for determining if a subject is at decreased or increased risk of having diabetes and/or pathology related to WRS according to the invention, are characterized in that said diabetes and/or pathology related to WRS is selected from the group consisting of type 1 diabetes, type 2 diabetes, the others forms of diabetes, osteoporosis, arthritis, hepatic dysfunction, nephropathies and other renal dysfunction and mental retardation.

In a more preferred embodiment, said methods or kits according to the invention are characterized in that said diabetes and/or pathology related to WRS is selected from the group consisting of type 1 diabetes, type 2 diabetes and the others forms of diabetes.

In a particularly more preferred embodiment, said methods or kits according to the invention are characterized in that said diabetes and/or pathology related to WRS is type 1 diabetes.

The invention also relates to the use of cells, a mammal or a polypeptide according to the invention, for studying the expression and the activity of EIF2AK3 protein, and the direct or indirect interactions between said EIF2AK3 gene or their expression product and the chemical or biochemical compounds which may be involved in the activity of said EIF2AK3 gene or their expression product.

The invention also relates to the use of a cell, a mammal or a polypeptide according to the invention for the screening of chemical or biochemical compounds capable of interacting directly or indirectly with the EIF2AK3 protein, and/or capable of modulating the expression or the activity of said EIF2AK3 protein.

Also included in the invention are the methods for selecting a chemical or biochemical compound capable of interacting, directly or indirectly, with the EIF2AK3 protein, and/or allowing the expression or the activity of the said EIF2AK3 protein to be modulated, characterized in that it uses a cell, a mammal or a polypeptide according to the invention.

The chemical or biochemical compounds, characterized in that they are capable of interacting, directly or indirectly, with the EIF2AK3 protein, and/or allowing the expression or the activity of the said EIF2AK3 protein to be modulated, also form part of the invention.

Compounds characterized in that they are selected by a method according to the invention, also form part of the invention.

In a preferred embodiment, the present invention comprises the compound according to the invention, characterized in that it allows:

a modulation of the level of EIF2AK3 protein expression; and/or

an increase of pancreatic β-cells or integrity thereof; and/or

the prevention or treatment of diabetes and/or pathology related to WRS.

In a preferred embodiment, the present invention comprises a compound according to the invention, characterized in that it is chosen from an antibody, a polypeptide, a vector or a sense or anti-sense nucleic sequence according to the present invention.

The present invention further relates to compound according to the invention as a medicament, particularly for the prevention and/or treatment of diabetes and/or pathology related to WRS.

In a preferred embodiment, the present invention further relates to compound according to the invention as a medicament for the prevention and/or treatment of diabetes and/or pathology related to WRS, characterized in that said diabetes and/or pathology related to WRS is selected from the group consisting of type 1 diabetes, type 2 diabetes, the others forms of diabetes, osteoporosis, arthritis, hepatic dysfimction, nephropathies and other renal dysfimction and mental retardation.

In a more preferred embodiment, the compounds according to the invention are characterized in that said diabetes and/or pathology related to WRS is selected from the group consisting of type 1 diabetes, type 2 diabetes and the others forms of diabetes.

In a particularly more preferred embodiment, the compounds according to the invention are characterized in that said diabetes and/or pathology related to WRS is type 1 diabetes.

### LEGENDS TO FIGURES

FIGS. 1A, 1B and 1C. Results from characterization of extended sets of microsatellite markers in three regions of potential linkage to WRS:

FIG. 1A: region 1 (chromosome 2) in families WRS1 and WRS2;

FIG. 1B: region 2 (chromosome 2); and

FIG. 1C: region 3 (chromosome 9) in family WRS1.

The regions 2 and 3 have been rejected as unlikely to be of interest because the parental haplotypes transmitted to WRS patients were not identical throughout (FIGS. 1B and 1C).

The marker order was obtained from public databases, and was revised when necessary according to observed recombinants in these families. For some closely linked markers, the order remains ambiguous, because of limited information or discrepancies between publicly available data.

FIGS. 2A and 2B. EIF2AK3 variations in WRS patients. Sequence chromatograms are shown for the regions of the variation in the index patients and normal controls from the WRS1 family (FIG. 2A: 1103-insT) and the WRS2 family (FIG. 2B: 1832G>A). The corresponding genotypes were determined by sequencing genomic DNA (both families) and a PCR-RFLP assay (WRS1). In the WRS2 family, a frequent polymorphism at intron 10-811A/T is also visible in the sequence (index patient: T/T; control: A/A).

FIGS. 3A and 3B. Effect of EIF2AK3 variation from the WRS families at the protein level.

FIG. 3A. The variations in WRS1 and WRS2 families (ins345fs/ter345 and R587Q respectively) are shown. The signal peptide is colored in black, the regulatory domain is indicated by hatched lines, and the catalytic domain is uncolored.

FIG. 3B. Amino-acid sequence conservation is shown around the R587Q variation for EIF2α kinases and a related kinase (WEE1) from different organisms (SEQ ID NOS 110-132, respectively, in order of appearance). Sequence alignments were performed using the BLAST program. Exact conservation are indicated in red, conservative changes are indicated in yellow, and non-conservative changes are uncolored. The kinase subdomains I and II (partial) are indicated.

### EXAMPLE 1

### Methods

Clinical Diagnosis and Families

Two families were studied. WRS1 was of Tunisian origin and has been reported earlier ³⁹, while WRS2 was of Pakistanese origin. Diabetes was of early onset in all patients (2^{nd} to 6^{th} month of life), while epiphyseal dysplasia and/or delayed growth was diagnosed in the first two years of life in the two living patients of WRS1 and in the older patient of WRS2, but was not diagnosed in the other affected children because of early death (age 5 months, in WRS1) or young age of the patient (age 6 months, last child of WRS2). All biological samples collections and examinations were done with informed consent from the families. Venous blood samples were collected on EDTA for DNA extraction for all family members, except for the first child from family WRS1, where DNA sample was extracted from an autopsy liver sample. Fresh blood samples from parents and one child of WRS1 family was also collected on FDTA for RNA extraction.

Genotype Characterization

Genotype characterization of microsatellite polymorphisms was performed using fluorescent-labeled primers on ABI377 sequencers as described (Dib, C. et al., Nature, 380, 152-154, 1996).

Linkage Analysis

Two-point linkage analyses were performed using the program LODSCORE from the LINKAGE package (Lathrop, G.M., et al., Proc. Natl. Acad. Sci., U.S.A, 81, 3443-6, 1984), and multipoint analyses using the LINKMAP program. We assumed a fully penetrant recessive disease, with a disease gene frequency of 0.00001. Allele frequencies from the CEPH parents (ftp://ftp.genethon.fr/pub/Gmap/Nature-1995/alleles/) or deduced from information available in public databases were assumed for the analyses. For the genome screening, we assumed genetic distances in Kosambi cM as estimated on the Marshfield map, available on the GDB web site (http://www.gdb.org). In the fine mapping of WRS gene, the marker order was obtained from public databases, and recombination events observed in WRS families. Calculations of the probability of obtaining maximum evidence of linkage with a fully-informative marker locus were made with the program SLINK. he 5′ region of the gene, was identified by means of its sequenced BAC ends

Genomic Structure of EIF2AK3 Gene

A full-length human cDNA corresponding to EIF2AK3 was cloned previously (Genbank: AF110146) (Shi, Y., et al., J. Biol. Chem. 274, 5723-30, 1999). We identified a BAC clone clone covering the 3′ end of EIF2AK3 (CIT978SK-121D7) by screening the Caltech BAC library with primers located in the 3′ UTR of the gene (STS3′F: GGGGCATAACCTAATTTGAGC (SEQ ID NO: 16) and STS3′R: GGGGACTTTCCTTCTTCTGC (SEQ ID NO: 17)). An overlapping BAC, RCPI-11-349C16, covering the 5′ region of the gene, was identified by means of its sequenced BAC ends (Genbank: AQ543 111) by blast search using EIF2AK3 cDNA. The intron/exon structure of the gene (Table 1) was established by sequencing long PCR fragments obtained by inter-exons PCR and comparison to the reference cDNA sequence (AF 110146), and by complementary direct sequencing on BAC clones DNA using exonic primers.

TABLE 1

Genomic structure of EIF2AK3

| | position | | |
|---|---|---|---|
| Exon | on cDNA | Acceptor site | Donor site |
| 1 | ..1-377 | ... | CGCGCGGCAGgtgaggggctgccga |
| 2 | 378-507 | ttttaatatttacagGTCATTAGTA | CAAACCAGAGgtaagaattttctgt |
| 3 | 508-702 | tagcttctgttttagGTATTTGGGA | TAGTGGAAAGgtaagtgaaaatgct |
| 4 | 703-836 | gtcttttcaggtgagGTGAGGTATA | GCAATGAGAAgtgtgtattcagata |
| 5 | 837-1071 | ctttgtaaatttaagGTGGAATTTC | GGAGTACCAGgtacctaacaccact |
| 6 | 1072-1234 | tccatttttgtttagTTTTGTACTC | GTTTACTTGGgtgagtaaatgtatc |
| 7 | 1235-1375 | ttctggttgattcagGAATGTATAG | CCCTTAATTCgtaagtgaattgtaa |
| 8 | 1376-1498 | ataattttcttttagATTCTCCTTC | TATCCATATGgtaagtgaaaatact |
| 9 | 1499-1719 | tgtttctatttgaagATAATGGTTA | TCCTCACAGGgtaagaatcatggtt |
| 10 | 1720-1832 | ttttcaccttatcagCAAAGGAAGG | ATATATCACGgtaagagtcttataa |
| 11 | 1833-1955 | tatctctttttaaagATATCTAACT | TCCCCAATAGgtaatgggtggtacc |
| 12 | 1956-2105 | ttttctctctttcagGGAATTGGCT | AAGATGAAAGgtaactaactttgtt |
| 13 | 2106-2886 | ttctcccacttttagCACAGACTGG | GGACCTCAAGgtctgtatttgtgga |
| 14 | 2887-3054 | ttgtattctttccagCCATCCAACA | CCCAGAGCAGgtgagtttttcagac |
| 15 | 3055-3156 | tatgtgggatttcagATTCATGGAA | GAGAGTCAGGgtaagtaccctccct |
| 16 | 3157-3219 | tttttttctttttagACCTTAACTG | TCGTTGTGAGgtatgtgtaattctc |
| 17 | 3220-end | tttttatattttcagTACGTGATGG | ... |

Legend of Table 1:
cDNA used as reference: Genbank AF110146. Intronic sequences are in lower cases and exonic sequences in upper cases. AG and GT splice consensus sequences are underlined.

Mutation/polymorphism Screening and Haplotype Estimation

Mutation screening was performed in an WRS1 index patient and his two parents, with a normal Caucasian individual used as a control, by direct sequencing of the coding regions of the cDNA on RT-PCR amplified product, and on an WRS2 index patient and his father, with a normal Caucasian individual used as a control, by sequencing coding regions of the gene on PCR-amplified genomic DNA. Cosegregation of the mutation identified in WRS1 family with WRS was confirmed on genomic DNA by PCR-RFLP method using primers PEK1: CTGACTGGAAAGTTATGG (SEQ ID NO: 18) and PEK2: AAAAGACTGATGGGAATGAC (SEQ ID NO: 19) followed by a restriction enzyme digest with AflII. After digest, the normal allele gives 302 and 35 bp fragments (presence of the restriction enzyme site), while the mutant allele gives a 337 bp fragment (no site), which were resolved by agarose gel electrophoresis. Screening for polymorphisms was performed by sequencing all the exons of the gene on PCR-amplified genomic DNA in unrelated healthy Caucasians. RNA extraction on fresh blood was done using QIAmp RNA blood mini-kit (Qiagen), and RT-PCR using the ProSTAR single-tube RT-PCR system (Stratagene). Sequencing reactions were performed on an ABI3700 sequencer, using the big-dye terminator chemistry (Rosenblum, B. B., et al., Nucleic Acids Research, 25, 4500-4 1997 ; Heiner, C. R., et al., Genome Research, 8, 557-61, 1998), using one of the template primers as sequencing primer.

Haplotypes frequencies were estimated for the eight polymorphic sites with allele frequencies >0.05 from the genotype data on the 95 unrelated Caucasian controls. This was done by a step-wise procedure to reduce the number of haplotype combinations that needed to be considered. In the first step, two sites were arbitrary chosen, and maximum likelihood estimates of the haplotype frequencies were obtained assuming Hardy-Weinberg equilibrium. The estimates were found using an EM algorithm starting from initial frequencies calculated under the assumption of linkage disequilibrium. Then, another site was chosen arbitrarily, and the corresponding haplotype frequencies for all three sites were estimated in the same way, starting from initial values based on the previous haplotype frequencies estimates for the first two sites, and the assumption of linkage equilibrium with the new site. This procedure was continued until all sites were included in the estimates. Thus, whenever a haplotype had a frequency estimate of 0 at one step, all haplotypes involving the same combination of alleles had 0 frequency at later stages.

Genbank Accession Numbers

Human EIF2AK3 cDNA sequence: AF110146 ; EIF2AK3 protein sequences: AAD19961 (human), AAD03337 (mouse), AAC83801 (rat). GCN2 and related kinases protein sequences: P15442 (yeast), CAB58363 (mouse), CAA92117 (C. elegans), AAC13490 (D. melanogaster), CAB60699 and CAB11253 (S. pombe); HRI protein sequences: AAF18391 (human), Q9Z2R9 (mouse), Q63185 (rat), P33279 (rabbit) WEE1 protein sequences: AAB60401 (human), (P47810) mouse, (BAA06624) rat, (AAD52983) Z. mais, (AAF02869) A. thaliana, (AAC46913) D. melanogaster, (P47817) X. laevis; PKR protein sequences: AAC50768 (human), Q03963 (mouse), AAA61926 (rat); EIF2AK3 genomic DNA sequences generated in this study: Genbank numbers to be assigned.

### EXAMPLE 2

### Genome Screening in a WRS Family

Initially, a consanguineous family (WRS1) of Tunisian descent with four children has been studied, three of whom were affected with WRS, and one who was healthy. The parents were first cousins and both were unaffected. It has been calculated that a maximum lod score of 2.53 could be obtained with complete information on identity-by-descent under a model assuming a rare recessive mutation that has entered the pedigree once and been inherited by both parents from one of their grandparents. Simulation studies indicated that with markers giving full information on identity-by-descent, the frequency of observing the maximal lod score by chance in a region that is unlinked to the trait would be approximately 0.6%, i.e. it has a nominal p-value of 0.006. As described below, one such region of potential linkage was identified in the WRS1 family in a genome-wide linkage study. Subsequently, linkage to this region was confirmed in a second family collected during the course of the study.

Genome-wide linkage was undertaken with 289 microsatellite markers from the Genethon screening set (http://www.genethon.fr). Eighteen of the markers were on chromosome 15 (mean spacing: 6.3 cM) and the remainder was distributed over the other autosomes (mean spacing: 13.1 cM). Twenty-four additional microsatellites were added at a later stage as a complement to markers that were insufficiently informative in the original screen. These were markers that were either homozygous in both parents, or heterozygous in one parent with evidence of complete linkage to WRS in meioses from that parent. Linkage analysis was performed under the assumption that the trait was due to a rare recessive mutation with complete penetrance and no phenocopies. Marker allele frequencies were estimated from the CEPH families.

Four markers were fully informative and had patterns of transmission that were consistent with complete linkage to the trait (see Table 2).

TABLE 2
Linkage results for selected regions in the family WRS1.

| Region | locus | d | | Lod score |
|---|---|---|---|---|
| 1 | D2S380 | 10.2 | 0.194 | 0.524 |
| | D2S286 | 17.2 | 0.001 | 0.884 |
| | D2S113 | 11.7 | 0.001 | 2.481 |
| | D2S160 | 19.7 | 0.001 | 1.851 |
| | D2S112 | 9.4 | 0.243 | 0.164 |
| | D2S151 | 17.4 | 0.682 | 0.045 |
| | D2S382 | 16.8 | 0.750 | 0.118 |
| 2 | D2S364 | 12.4 | 0.001 | 1.993 |
| | D2S116 | — | 0.105 | 0.660 |
| 3 | D9S168 | 2.2 | 0.001 | 1.920 |
| | D9S269 | — | 0.001 | 1.170 |

Legend of Table 2: Results are shown for the three chromosome region (two regions on chromosome 2 and one region on chromosome 9) for which the initial data were compatible with complete linkage to WRS. Markers indicated in bold were fullly informative for linkage and consistent with the hypothesis of no recombination with WRS. d: distance to next marker (cM). θ: estimated recombination fraction with WRS locus.

Two of the markers were adjacent (at 12 cM distance) in a single region on chromosome 2; another of the markers was also on chromosome 2 but separated from the first two by distance of 63 cM; the fourth marker was on chromosome 9. The two adjacent markers on chromosome 2, D2S1 13 and D2S160, were completely linked to the trait, and gave lod scores of 2.48 and 1.85 respectively at θ=0. Another marker, D2S286, nearby these two was partially informative and gave a lod score of 0.884 at θ=0. The multilocus lod score of the three markers from this region was the maximum possible from the pedigree (2.53).

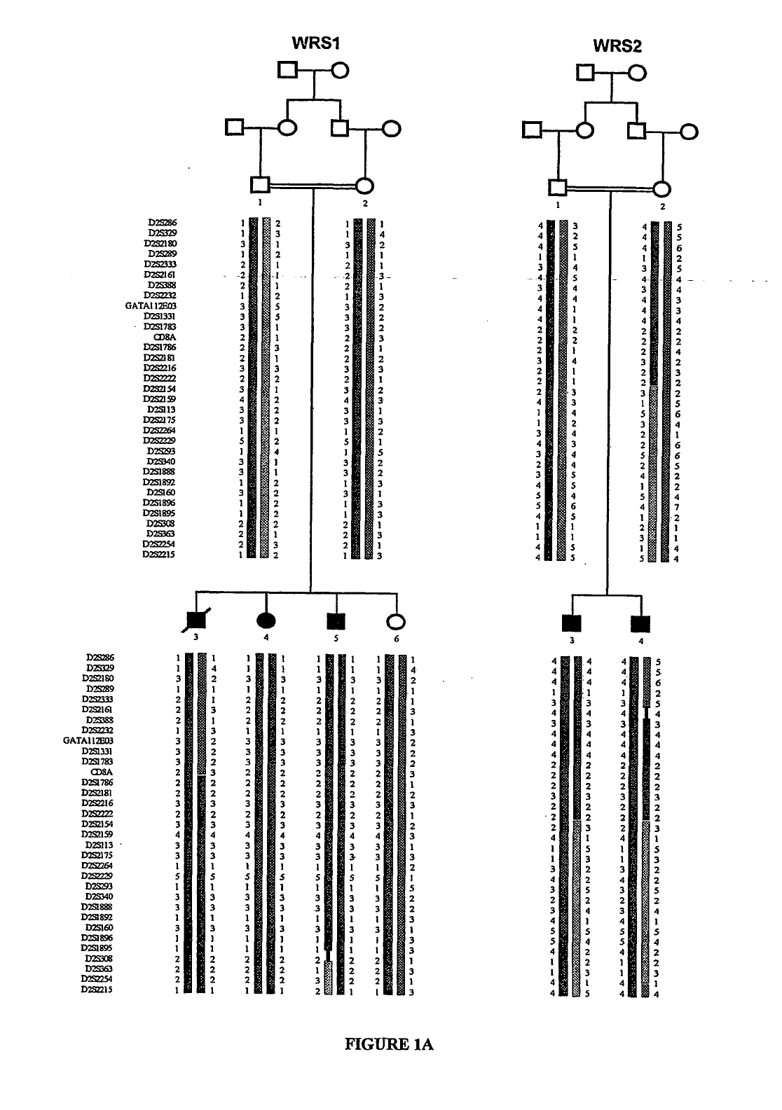

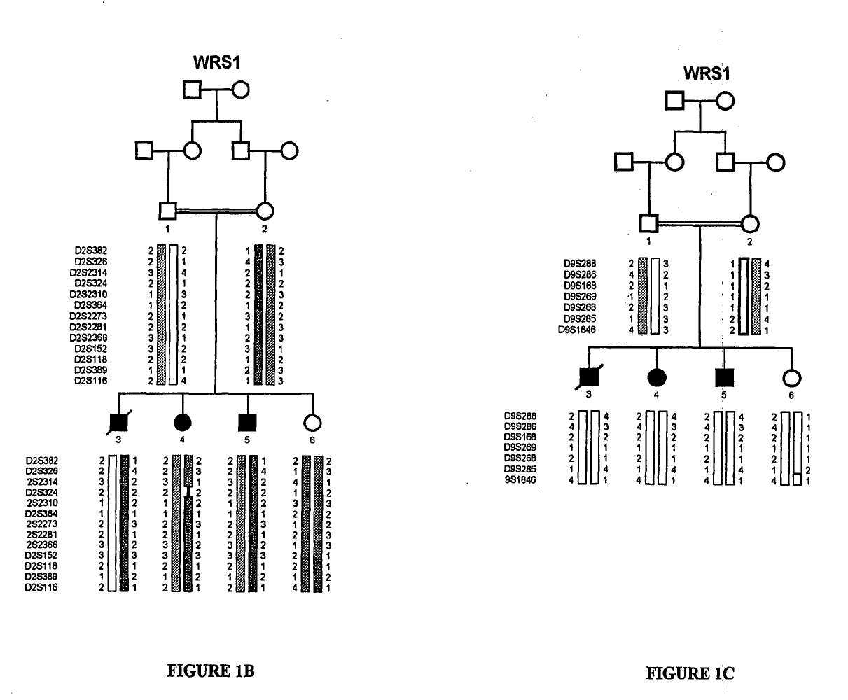

In the second region on chromosome 2, the marker D2S364 gave a lod score of 1.19 at θ=0. On chromosome 9, D9S168 showed a pattern consistent with linkage with a lod score of 1.92 at θ=0. This marker was added to the original genome-wide set to complement the linkage information from D9S269, which was informative for meioses from osne parent and gave a lod-score of 1.17 at θ=0.Other markers from these three regions were selected for genotyping in this family in order to provide more complete information on possible haplotype identity in these regions. To this end, 40 additional markers between D2S380 and D2S112 (region 1), 10 additional markers between D2S382 and D2S116 (region 2) and 3 additional marker between D9S288 and D9S1846 (region 3) were characterized. Inspection of the genotypes and frther statistical analysis provided evidence to confirm linkage in region 1, where the haplotypes transmitted to affected offspring carry identical microsatellite alleles in a 17 cM interval between CD8A and D2S363 (FIG. 1A). The other regions were rejected at this stage as unlikely to be of interest because the parental haplotypes transmitted to WRS patients were not identical throughout (FIGS. 1B and 1C).

### EXAMPLE 3

### Fine Mapping of the WRS Gene

During the course of the linkage study, a second WRS family (WRS2) with a different ethnic origin has been obtained. This family consisted of two affected children and their parents, who were unaffected and first cousins. A selection of microsatellite markers from region 1 (D2S380-D2S112) on chromosome 2 was characterized in this family. Two-point linkage analyses in the combined family panel gave lodscores of up to 3.41 at θ=0, and multilocus linkage analysis reached a maximnum lodscore of 4.33 at the position of markers D2S1786/D2S2181/DS2S2216/D2S2222 confirming linkage to WRS (data not shown).

Next, the overlap has been examined between the segments of potential identity-by-descent in region of linkage from the affected offspring in the two families in order to determine a smaller region in which the gene responsible for WRS should reside. The overlap consists of a segment of approximately 2-3 cM between CD8A and D2S2154 containing the four tightly linked microsatellite markers listed above (within˜1 cM). These are homozygous for all the affected offspring and exhibit complete linkage to WRS in both families (FIG. 1A). The critical region containing the gene is defined by two recombination events: one is an obligate recombination in individual WRS1-3 (defining the distal boundary between CD8A and D2S1786), and the other is a recombination inferred to have occurred in one of the meioses leading from the great-grandparents to the parents in family WRS2 (defining the proximal border between D2S2222 and D2S2154).

### EXAMPLE 4

### Mutation Screening of a Candidate Gene within the Region of Linkage: the Eukaryotic Translation Initiation Factor 2 Alpha Kinase 3 (EIF2AK3)

A partial physical map of the critical region was constructed from information in public databases. Of the several expressed sequence tags (ESTs) potentially mapped in the region, one (D2S 1994/WI-6863) that mapped to the Whitehead YAC contig WC2.7 attracted our interest. This EST was recently identified as the gene EIF2AK3 (Shi, Y., et al., 1999), a serine/threonine kinase and a major candidate gene for WRS because of its high level of expression in the pancreas islet, as well as in the placenta. Independent mapping of this gene to this region was also recently obtained by radiation hybrid mapping and in situ hybridization by Hayes et al. (Hayes, S. E., et al., Cytogenet Cell Genet, 86, 327-328, 1999). EIF2AK3 has been screened for mutations in index patients from families WRS1 and WRS2.

Since EIF2AK3 is expressed ubiquitously at low level (Shi, Y., et al., 1999), the coding region of the gene was scanned in an index patient from family WRS1 by direct sequencing of RT-PCR products generated on total RNA from fresh whole blood. As fresh blood samples were not available for the WRS2 family, the genomic organization of this gene for mutation screening has been also established (see Methods). The exonintron structure defined conformed fully with the published consensus splice sequences (Breathnach, R., et al., Annu. Rev. Biochem., 50, 349-383, 1981). Mutation screening in WRS1 and WRS2 families was performed in the coding regions of the gene, directly on the cDNA (family WRS1) or by amplifiing exons from the genomic DNA (family WRS2), using primers shown in Tables 3A and 3B.

Tables 3A and 3B. Sequence of primers used for mutation and polymorphism screening of EIF2AK3.

TABLE 3A

Primers used for sequencing EIF2AK3 cDNA

| Name of | | | PCR |
|---|---|---|---|
| primers | Forward primer | Reverse primer | product size |
| PEK_cDNA1 | GAGAGGCAGGCGTCAGTG | TTTCCATGCTTTCACGGTCT | 569 |
| PEK_cDNA2 | CCAGCCTTAGCAAACCAGAG | CTCCCATTCCAGATGTCCTC | 578 |
| PEK_cDNA3 | AAGGTTTCGGTTGCTGACTG | ATGTGGGTTGTCGAGGAATC | 585 |
| PEK_cDNA4 | GGAGAGGAACAAACGAAGCA | CATTGGGCTAGGAGAGCTGA | 610 |
| PEK_cDNA5 | AGACTGGCCACTCAGCTCTC | GTGAACTGGGCTGGAGTTTT | 599 |
| PEK_cDNA6 | TGTCCTCCAAGACCAACCAC | GCATGTCTTGAACCATCACG | 607 |
| PEK_cDNA7 | CCATTCAGCACTCAGATGGA | TGCAATTTTGGACAGGCATA | 372 |

TABLE 3B

Primers used for sequencing EIF2AK3 genomic DNA

| | | | PCR |
|---|---|---|---|
| Exon | Forward primer | Reverse primer | product size |
| 1 | GAGAGGCAGGCGTCAGTG | CGCGCGTAAACAAGTTGC | 403 |
| 2 | TGAGCATGTGGGATAAGTGC | TGCCCTAAAGGGACACAAAC | 333 |
| 3 | TCAGGATCAAGACTCCAGCTC | TGACAACCTCAGGGGAAAAT | 448 |
| 4 | GGAGTTGGTAATCTAACTGATGC | CCAACAGCAACATTATCTGAA | 328 |
| 5 | GCCCTCTTGTGGCATAAATC | CTGGGAGAGGAAGAACCGTA | 449 |
| 6 | TACTTGGGGCTCTCAGCTTG | GGGACTCCTGAAGTAGGAAGG | 374 |
| 7 | CCCTCCCTGTTTTTGTTGAA | GGGCAAAGACAGTCAGGATT | 389 |
| 8 | CTGGGCCATTTGTTTAACTT | TGAAATTGTCTCCCAAGATG | 384 |
| 9 | TAGTTAAAGACGGGCCTATT | CAAGAGTAGCTTTGGTGGAG | 396 |
| 10 | AAGACTGGAGGGATAGCAGT | AGATCTTAGGTCATTTCTTCTTTG | 371 |
| 11 | TGAACTGATTTTCACATTACCAC | AATTGGCAGCACTTAGAACC | 340 |
| 12 | GCCTTCAGGGTTGTCTTACT | CATTGTAATCACACAAGCAAA | 384 |
| 13 | ACAGAGGGTGCAGTTCAGGT | CACAATGGTTGCCAATATGC | 537 |
| 13 | AAGGTCAAGGGAGAGAACCT | ACCTCTGCTCTCAGATGCTT | 555 |
| 14 | CATGCACACCCACTGTACTT | CTGGAACACTACTGCCAGTTT | 348 |
| 15 | CTTTGGGATTCAATAATGCT | CCAATCTGCTGGTATTAAGAA | 288 |
| 16 | TGTGGAATCTGTGGGATGTG | TGCTAAGGACCGCTTACGTT | 356 |
| 17 | TTTTGCCAGCACTGATTTTA | TTTCAAGTCTGCAATTTTGG | 367 |
| 17* | CAACTCCCATAGCCCTTTGC | TAATTTACCCGCCAGGGACA | 502 |
| 17* | GAGGTAGCAGCAATCCCTAA | CATGGATTGATTTCAGAATTTTT | 626 |

Legend of Table 3B:
Primer pairs labeled with a star (*) cover non-coding sequences, and were used only to screen for polymorphisms in controls.

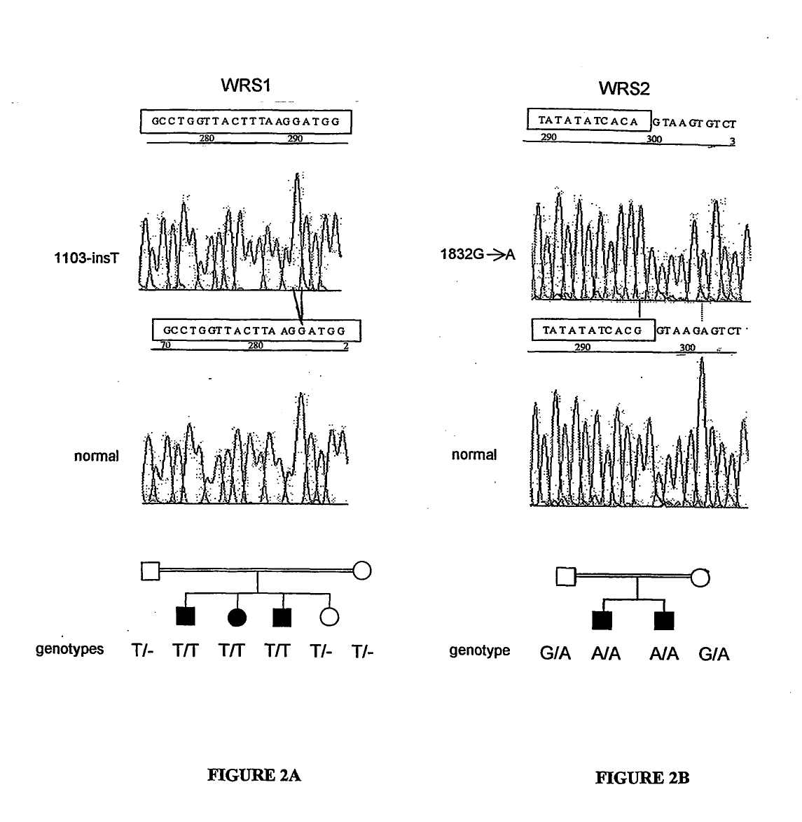

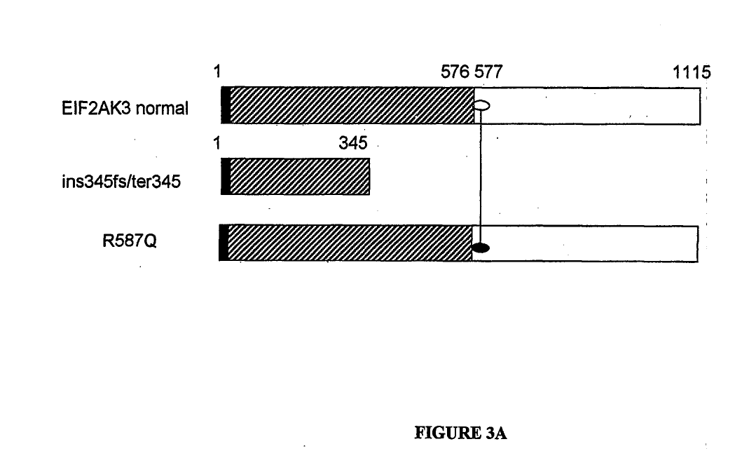

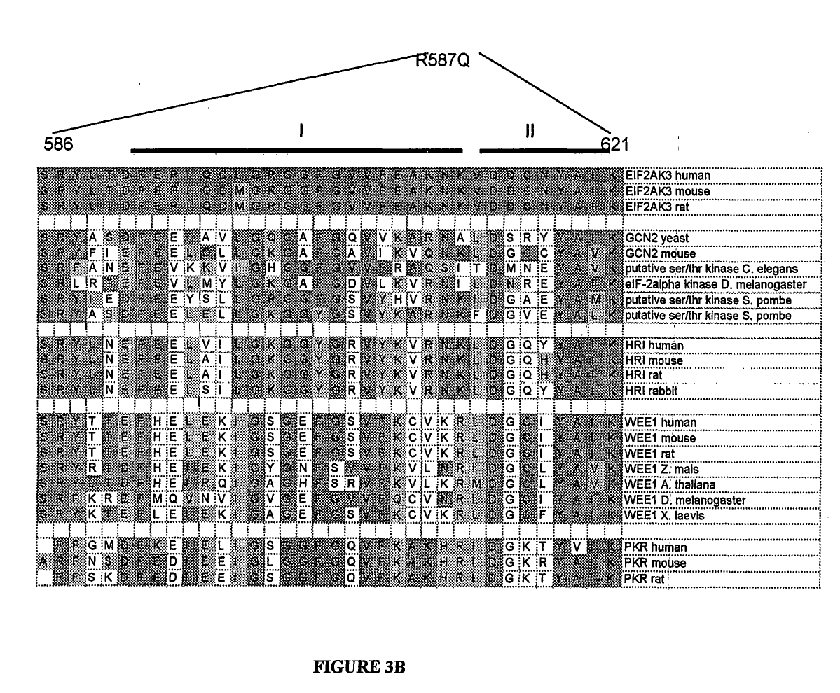

Two distinct mutations were identified in the two families, in the homozygous state in WRS patients, and heterozygote in their parents as well as in the healthy sib in family WRS1 (FIG. 2). Cosegregation of the mutation and the disease alleles was confined by direct sequencing of the relevant region of genomic DNA from all the family members (both mutations) as well as by a PCR-RFLP essay designed for scoring the presence/absence of the mutation in WRS1 (see methods). In addition, a microsatellite polymorphism identified in intron 15 of EIF2AK3 (Table 4A), was found to be fully informative in WRS1, and semi-informative in WRS2, and cosegregated with the disease alleles in both families (not shown). Both mutations were absent in 190 Caucasian, 95 Japanese and 95 Black African controls, as shown by direct sequencing (both mutations) and by a PCR-RFLP essay (mutation in WRS1 family). Description of the effect of the two mutations at the protein level is shown in FIG. 3. The WRS1 mutation is an insertion of a T at position 1103 (1103insT), which creates a frameshift at position 345 and premature termination of the protein at the same position (ins345fs/ter345). Thus, the WRS1 mutation produces a truncated protein that is devoid of part of the regulatory domain (amino-acid 1-576) and the totality of the catalytic domain (amino-acids 577-1115); this is likely to result in a complete loss of function of the protein.

Tables 4A, 4B and 5: Polymorphisms identified in EIF2AK3 oxons and flanking intronic regions, and estimated haplotype combinations and frequencies

TABLE 4A
EIF2AK3 frequent polymorphisms

| | | | genomic DNA | genomic DNA | |
|---|---|---|---|---|---|
| Intron/exon | CDNA position | amino acid | segment | position | Frequency |
| Exon 1 | 112-132(CTG)7/8 | 14-20(L)7/8 | PEK-ex1 | 112-132(CTG)7/8 | 0.75/0.25 |
| Exon 2 | 476C/G | 135Ser/Cys | PEK-ex2 | 1141C/G | 0.68/0.32 |
| Exon 3 | 566G/A | 165Arg/Gln | PEK-ex3 | 978G/A | 0.62/0.38 |
| Intron 10 | — | — | PEK-ex10 | 811A/T | 0.74/0.26 |
| Exon 11 | 1860G/A | 596Gln/Gln | PEK-ex11 | 707G/A | 0.30/0.70 |
| Exon 13 | 2179T/G | 703Ser/Ala | PEK-ex12-13 | 1638T/G | 0.68/0.32 |
| Intron 15 | — | — | PEK-ex15 | 845A/C | 0.94/0.06 |
| Intron 15 | — | — | PEK-ex16-17 | 1217-1255(CA)n | ND |
| Intron 15 | — | — | PEK-ex16-17 | 1641-1642[AT]/- | 0.95/0.05 |

Legend of Table 4A:
Polymorphisms are positioned relative to the reference cDNA sequence (Genbank AF110146), and to genomic sequences which have been generated

during the establishment of the intron/exon structure of the gene, with indication of amino-acid changes. Frequencies have been estimated by screening a population of 95 unrelated healthy Caucasians.

TABLE 4B
EIF2AK3 haplotypes

| | estimated |
|---|---|
| Haplotype | frequency |
| 7GAAAGAI | 0.310 |
| 7CGAGTAI | 0.300 |
| 8CGTATAI | 0.247 |
| 7CAAATAI | 0.058 |
| 7CGAATCD | 0.053 |
| 7CAAAGAI | 0.005 |
| 7GAAATAI | 0.005 |
| 7CGAATAI | 0.005 |
| 7CGAATCI | 0.005 |
| 8CGTGTAI | 0.005 |
| 7GGAGGAI | 0.006 |

Legend of table 4B:
<<7>> corresponds to the 7-repeat allele and <<8>> to the 8-repeat allele at the exon1 polymorphism. <<I>> and <<D>> correspond to the AT-insertion and -deletion alleles respectively in the last intron 15 polymorphism.

TABLE 5
EIF2AK3 less frequent polymorphisms

| genomic | | | | | Frequency |
|---|---|---|---|---|---|
| DNA | cDNA | cDNA | amino | | of the |
| segment | position | position | acid | Intron/exon | rare allele |
| PEK-PRO5 | 3640C/T | | | Promotor or non | 0.03 |
| | | | | translated 5′ domain | |
| PEK-ex2 | 1261G/T | | | Intron2 | 0.005 |
| PEK-ex3 | 887A/G | | | Intron2 | 0.005 |
| PEK-ex3 | 903A5/A6 | | | Intron2 | <0.005 |
| PEK-ex5-8 | 700G/A | 1008G/A | 312Ser/Ser | Exon5 | 0.005 |
| PEK-ex9 | 734G/A | 1680G/A | 536Thr/Thr | Exon9 | <0.005 |
| PEK-ex10 | 672T/A | | | Intron9 | 0.005 |
| PEK-ex10 | 739A/T | 1766A/T | 565Asp/Val | Exon10 | <0.005 |
| PEK-ex16-17 | 2832G/A | 3223G/A | 1051Val/Met | Exon17 | <0.005 |
| PEK-ex16-17 | 3329-3340-(TA)12/13 | 3720-3731- | | Exon17 | 0.02 |
| | | ins[TA] | | | |

The WRS2 mutation is a 1832 G to A transition, resulting in a Glutamine for Arginine mutation at position 587 (R587Q), located within the catalytic domain of the protein. Although this mutation is not located in a known functional site of the protein it is at the flanking border of the highly conserved kinase subdomain I, as defined by Hanks and Hunter (Flanks, S. K, et al., Faseb J., 9, 576-96, 1995), and is completely conserved between eIF-2α kinases from different organisms: EIF2AK3 from mouse and rat, GCN2 from S. cerevisiae and its homologs from different organisms, including S. pombe, C. elegans, D. melanogaster and mouse, HRI (eIF-2α kinase Heme Regulated Inhibitor) from human, mouse, rat and rabbit, PKR (Protein Kinase, interferon-inducible double stranded RNA dependant) from human, mouse and rat. In addition, the region is highly conserved in WEE1, another kinase whose catalytic domain shares homology with these ser/thr kinases and its homologs from different organisms (FIG. 3). Interestingly, a single amino acid change at a similarly conserved position, Alanine for Lysine at position 614 in rat EIF2AK3 (corresponding to position 621 in human EIF2AK3 as shown in FIG. 3) results in a complete loss of kinase activity (Shi, Y., et al., 1999).

### EXAMPLE 5

### Polymorphisms Screening in EIF2AK3

Because of the multiple pathological manifestations that characterize WRS, including insulin-dependent diabetes, it has been decided to screen the totality of the exons for polymorphisms in a panel of 95 normal Caucasian individuals. These variants and knowledge of possible haplotypes will constitute a valuable resource for testing the implication of this gene in several disorders, including diabetes or growth disorders. A total of eight variants located in exons or close flanking intronic regions were identified, with rare allele frequency greater than 0.05 (Table 4A). Five of these variants map in the coding region of the protein, and four of them affect the anino-acid sequence. The data were consistent with the arrangement of the eight variants on 11 haplotypes, five of which had estimated frequencies greater than 0.05 (Table 4B). In addition, additional rare variants were identified, which occurred in one or two alleles out of the 190 characterized (Table 5). A microsatellite based on a (CA)n tandem repeat in intron 15 was also identified, and confirmed to be polymorphic (not shown), and is also listed in Table 4A. This polymorphism will be of interest for family studies exploring the role of EIF2AK3 in diseases.

These results demonstrate that variations in EIF2AK3 gene are responsible for WRS and their related pathologies in two consanguineous families of different ethnic origins.

These results provide strong evidence for the role of EIF2AK3 in WRS, and its involvement in the etiology of insulin-dependent diabetes and other features of the syndrome.

This is a key finding for understanding the molecular mechanisms that could explain diabetes, skeletal dysplasia and other manifestations of WRS. EIF2AK3 plays a role in the regulation of protein translation (Shi, Y., et al., Mol. Cell. Biol., 18, 7499-509 1998 ; Harding, H. P., et al., Nature, 398(6722):90, 1999, Mar. 4, Nature 397, 271-4 1999), and is highly expressed in the pancreatic islet cells (Shi, Y., et al., J. Biol. Chem. 274, 5723-30, 1999). Based on our study, EIF2AK3 appears to have an important function in maintaining the integrity of pancreatic β-cells.

EIF2AK3 is a recently identified member of the eIF-2α kinase family, which also includes the heme-regulated inhibitor kinase (HRI), the double stranded RNA dependant protein kinase (PKR) and the Yeast GCN2 (Shi, Y., et al., 1998 ; Harding, H. P., et al., 1999). Interestingly, one of these related genes, PKR, has been shown to play a role in the control of cell growth and apoptosis (Srivastava, S. P., et al., J. Biol. Chem., 273, 2416-23 1998), raising the possibility that EIF2AK3 has similar functions that could be relevant to the characteristic -pancreas ,β-cell absence in WRS patients. In addition, from its high level of expression in the pancreatic islet cells, EIF2AK3 is a good candidate to have a role in the fine regulation of insuin expression in response to glucose, a rapid process which takes place at the level of protein synthesis (Goodison, S., et al., Biochem. J., 285, 563-8, 1992; Gilligan, M., et al., J. Biol. Chem., 271, 2121-5, 1996).

Various lines of evidence and hypotheses can be evoked to explain the role of EIF2AK3 in both diabetes and bone disorders. Variation in genes expressed in the chondrocytes are known to be responsible for a number of bone disease that are similar to that observed in WRS (achondrogenesis/epiphyseal dysplasia/achondroplasia/hypochondroplasia/osteoporosis/arthritis). For example, gain-of-function mutations at FGFR3, which is also strongly expressed in pancreatic islet cells (Hughes, S. E., J. Histochem. Cytochem., 45, 1005-19, 1997), are responsible for achondroplasia and hypochondroplasia (Rousseau, F., et al., Nature, 371, 252-4, 1994 ; Rousseau, F., et al., J. Med. Genet. 33, 749-52, 1996). In contrast to the dominant effect of mutations at FGFR3 and other genes implicated in these disorders, the WRS phenotype is recessive. Thus, EIF2AK3 may exert a negative control on specific protein(s) from the pancreas and/or the chondrocytes, insuring adequate development and function of these organs under normal conditions, while in WRS patients, loss of functional EIF2AK3 could yield to over-expression of this (these) protein(s) and create the observed phenotypes associated with different organs. This hypothesis is consistent with the down-regulatory effect of EIF2AK3 on the level of protein translation. Since EIF2AK3 is expressed in the placenta, embryonic development may remain normal, because of the expression of the maternal EIF2AK3, while the post-natal growth and development processes affected by these mechanisms would be altered. Further studies on EIF2AK3 at the molecular level are required to test this hypothesis, and in particular, to identify the target protein(s) whose regulation may be directly affected by EIF2AK3 in this model.

Although diabetes in WRS does not appear to have an autoimmune etiology, the fact that the patients are permanently insulin-dependant diabetics suggests that the biological process involved in the syndrome could be of relevance to typical autoimmune insuin-dependent diabetes mellitus (IDDM) in conjunction with other genetic factors, notably in the MHC and the insulin gene. Recently, a novel gene, WFS1, has been shown to be responsible for another Mendelian syndrome (Wolfram syndrome) involving juvenile-onset insulin-dependent diabetes that is also characterized by loss of pancreatic β-cells, and it was speculated that such genes might be important in modulating susceptibility to IDDM (Inoue; H., et al., 1998). In a preliminary investigation of microsatellite markers and EIF2AK3 variants in multiplex IDDM families from France and the USA, we failed to detect significant evidence of a relationship to IDDM susceptibility (data not shown). However, this could be due to the limited size of the family sample explored, the presence of several risk variants in the gene, some of which may be rare, or their location in regulatory or intronic regions that have not been covered in our study. Evidence of linkage to this particular region has not been reported in other studies of IDDM (Hashimoto, L., et al., Nature, 371, 161-164, 1994 ; Davies, J. L., et al., Nature, 371, 130-136, 1994; Mein, C.A., et al., Nature Genet, 19, 297-300, 1998 ; Concamion, P., et al., Nature Genet, 19, 292-296, 1998) and non-insulin-dependant diabetes (Hanis, C. L., et al., Nature Genet, 13, 161-6, 1996 ; Pratley, R. E., et al., J. Clin. Invest., 101, 1757-64, 1998). The polymorphisms described here will allow direct testing of EIF2AK3 in patients and families from different sources to confirm the issue of its involvement with common forms of diabetes.

Although no evidence of clustering of autoimmune diseases loci has been reported for this region in human to date (Becker, K. G., et al., Proc. Natl. Acad. Sci. U.S.A., 95, 9979-84, 1998), it is remarkable that independent studies of autoimmune and inflammatory diseases in the mouse and in the rat have mapped several susceptibility genes for these diseases to the region of synteny to EIF2AK3 (Kawahito, Y., et al., J. Immunol., 161, 4411-9, 1998). In particular, a susceptibility locus has been earlier mapped for insulin-dependent diabetes in the mouse to this region (de Gouyon, B., et al., Proc. Natl. Acad. Sci., U.S.A., 90, 1877-81, 1993), and loci for several models of arthritis have been mapped to this region: collagen-induced arthritis (CIA) in the rat (Remmers, E. F., et al., Nature Genet, 14, 82-5, 1996) and in the mouse (Yang, H. T., et al., J. Immunol. 163, 2916-21, 1999), mycobacteria-induced arthritis (AIA) in the rat (Kawahito, Y. et., 1998), and pristane-induced arthritis (PIA) in the mouse (Vingsbo-Lundburg, C., et al., Nature Genet, 20, 401-4, 1998). However, because of the large number of autoimmune disease loci which have been mapped in several model organisms to date, and the large confidence intervals associated with these loci in most cases, interpretation of comparative mapping results of such disease susceptibility genes requires caution, and furlier studies will be required to evaluate the implication of EIF2AK3 in these particular disease models.

These findings may also be relevant to understand the other pathologic manifestations observed in WRS. In particular, WRS patients suffer from early renal complications, leading to nephropathy, and this gene therefore represents a good candidate for diabetic nephropathy. Examination of variants of this gene in osteoporosis in diabetics, whose occurrence is greater than in the non-diabetic population, will also be of interest.

### EXAMPLE 6

### Implication of EIF2AK3 in Type I Diabetes (T1DM)

Evidence of Linkage of Microsatellite Located in the Region of EIF2AK3 with Diabetes

In the above-presented examples, evidence has been shown that mutations at the EIF2AK3 gene are responsible for the Wolcott-Rallison syndrome. This syndrome associates in particular permanent neonatal or early-infancy onset insulin-dependant diabetes and multiple epiphyseal dysplasia, strongly suggesting that this gene may be involved in more frequent forms of diabetes, including type I diabetes (T1DM) and type II diabetes (T2DM).

As previously discussed, several groups have performed genome-wide screening of multiplex type I diabetes families, in order to map susceptibility genes for T1DM. In these published results on T1DM, and in genome-wide screening performed in T2DM as well, there was no evidence of linkage of microsatellite markers located near the EIF2AK3 gene (chromsome region 2p12) with diabetes, suggesting that the contribution of genetic variations at this gene to diabetes may be minor, or may be missed. There are several possible reasons for the lack of detection of genetic effects, such as the limited power of studies due to small sample sizes, the multiplicity of minor genetic effects contributing to the susceptibility to diabetes, and genetic and environmental heterogeneity within and between populations.

In this example, complementary information supporting an implication of EIF2AK3 in type I diabetes is presented.

This example particularly shows the evidence of linkage of microsatellite located in the region of EIF2AK3 with diabetes in some population groups.

A genome-wide search in the Scandinavian population, a population group which is thought to be relatively homogeneous in their environment and genetic background, has been completed. The family panel comprised 426 multiplex families and 485 affected sibpairs from Denmark, Sweden and Norway (ECIGS consortium—European Consortium for Insulin-dependant diabetes Genome Scan).

Genome wide screening was performed using 314 microsatellite markers located over the whole genome (average inter-marker spacing 11 cM). Complementary microsatellite markers typing were performed to increase the density of markers near EIF2AK3. Linkage analyses were performed using the ANALYZE program for single-point analysis (J. Terwilliger, Program SIBPAIR: sibpair analysis on nuclear families, ftp://linkage.cpcm.columbia.edu), and ASPEX program (E. Hauser, et al., Genet Epidemiol. 13, 117-37, 1996 ; D. Hinds and N. Risch, The ASPEX package: affected sib-pair mapping, ftp://lahmed.standford.edu/pub/aspex) for multipoint analyses.

In addition, analyses has been conducted in subsets of diabetic sibpairs that were both DR3/DR4 heterozygous (high risk HLA group), and in diabetics that did not share DR3 nor DR4 alleles (ow risk HLA group). Linkage results are shown in Table 6 below.

TABLE 6
Lod-score values at microsatellite markers near EIF2AK3
in single-point and multi-point analyses

| Marker locus | all | DR3/4 |
|---|---|---|
| D2S388 | 1.81 | 2.76 |
| D2S113 | 2.49 | 4.26 |
| Multi-point | 2.51 | 2.56 |

All: all diabetic sib-pairs
DR3/4: diabetic sib-pairs both HLA-DR3/4

Suggestive evidence of linkage was found in single-point analyses (lod-score up to 2.49), and in multipoint analysis oed-score peak at 2.51, in the interval D2S388-D2S 113). No evidence of linkage was found in the low risk HLA group (not shown), but increased evidence of linkage was found in the high risk HLA group (lod-score up to 4.26 in single-point analysis).

These complementary results support the hypothesis that variants at EIF2AK3 or at another gene located near EIF2AK3 are involved in the susceptibility to type I diabetes in the Scandinavian population. Because mutations in EIF2AK3 are able to generate a form of insulin-dependant diabetes, we favor the hypothesis that EIF2AK3 is the gene responsible for the linkage detected at microsatellite markers located in this regions of chromosome 2p12.

### EXAMPLE 7

### Third WRS Mutation

A third mutation has been identified which is present in the homozygous state, in an African patient with Wolcott-Rallison syndrome, whose DNA was provided by Dr. Catherine Diatloff((Hôpital Necker, Paris).

This third mutation is a missense mutation, located into exon 12:

position of the third mutation on the cDNA reference sequence (Genbank number: AF110146): 2037T>A (T=normal, A=mputated).

Amino Acid Change on the Reference Protein Sequence

655N (Asn) >K(Lys) (N=normal, K=mutated)

This amino acid change is located within the kinase subdomain IV, and presumably results in a protein whose function is dramatically altered.

(FIG. 2B: 1832G>A). The corresponding genotypes were determined by sequencing genomic DNA (both families) and a PCR-RFLP assay (WSR1). In the WRS2 family, a frequent polymorphism at intron 10-811A/T is also visible in the sequence (index patient: T/T; control: A/A).

1. Isolated variant nucleic sequence of a mammal genomic sequence of the gene coding for the translation initiation factor 2 alpha kinase 3 (EIF2AK3), said EIF2AK3 protein having the sequence SEQ ID No. 2, characterized in that the presence of said variant sequence in a mammal is capable of inducing the Wolcott-Rallison syndrome (WRS) or affects the risk of onset or progression of diabetes and/or pathology related to WRS.

2. Isolated variant nucleic sequence according to claim 1, characterized in that said diabetes and/or pathology related to WRS is selected from the group consisting type 1 diabetes, type 2 diabetes, the others forms of diabetes, osteoporosis, arthritis, hepatic dysfunction, nephropathies and other renal dysfunction and mental retardation.

3. Isolated variant nucleic sequence according to claims 1 and 2, characterized in that said diabetes and/or pathology related to WRS is selected from the group consisting of type 1 diabetes, type 2 diabetes and the other forms of diabetes.

4. Isolated variant nucleic sequence according to claims 1 to 3, characterized in that said diabetes and/or pathology related to WRS is linked to major decrease of pancreatic β-cells or integrity thereof.

5. Isolated variant nucleic sequence according to claims 1 to 4, characterized in that said diabetes and/or pathology related to WRS results from the alteration of the control which is exerted by EIF2AK3 on a specific protein from the pancreas and/or from the chondrocytes, said control, if normally exerted, insuring the adequate development and function of these organs.

6. Isolated variant nucleic sequence according to claims 1 to 5, characterized in that said variant sequence comprises a sequence selected from the group consisting of the sequences SEQ ID No. 3 to No. 15 or fragment thereof, provided said isolated variant nucleic sequence according to claim 1 is not the sequence SEQ ID No. 1.

7. Isolated variant nucleic sequence according to claims 1 to 6, characterized in that the protein EIF2AK3 encoded by said variant sequence presents at least one point variation compared to the sequence SEQ ID No. 2 of EIF2AK3.

8. Isolated variant nucleic sequence according to claims 1 to 7, characterized in that the protein EIF2AK3 encoded by said variant sequence presents a premature termination or at least one point variation in the catalytic domain aa 576-aa 1115 of the protein EIF2AK3 having the sequence SEQ ID No. 2.

9. Isolated variant nucleic sequence according to claims 1 to 8, characterized in that said sequence comprises an insertion of a T at position 1103 or a G to A transition at position 1832 in the sequence SEQ ID No. 1.

10. Isolated variant nucleic sequence according to claims 1 to 8, characterized in that said sequence comprises at least one of the nucleic sequence polymorphisms which are defined in Tables 4 A and B, and in Table 5, column “cDNA position” and/or “genomic DNA position”.

11. Isolated variant nucleic sequence according to claims 1 to 10, characterized in that said sequence is chosen from a human nucleic sequence.

12. Complementary sequence of the variant nucleic sequence according to claims 1 to 11.

13. Polypeptide encoded by the isolated variant nucleic sequence according to claims 1 to 11, characterized in that its amino acids sequence presents at least one point variation compared to the sequence SEQ ID No. 2 of EIF2AK3.

14. Polypeptide according to claim 12, characterized in that it comprises at least one of the amino acid variations as listed in the column “amino acid” in Tables 4A and 5.

15. Isolated nucleic acid sequence, characterized in that it encodes a polypeptide according to one of claims 13 and 14.

16. Isolated nucleic acid sequence, characterized in that it is selected from the group consisting of:
a) a fragment of nucleic sequence according to one of claims 1 to 12, and 15 comprising at least 12 bases;
b) a nucleic sequences capable of hybridizing specifically with the nucleic sequence as defined in a)and comprising at least 12 bases.

17. Isolated nucleic acid sequence according to claim 16 as a primer or a probe.

18. Isolated nucleic acid sequence according to claims 16 and 17, characterized in that it is selected from the group consisting of sequences SEQ ID No. 16 to SEQ ID No. 105.

19. Nucleic acid sequence which can be used as sense or anti-sense oligonucleotide, characterized in that its sequence is chosen from the sequences according to one of claims 16 and 18.

20. Cloning and/or expression vector containing a nucleic acid sequence according to one of claims 16 and 19.

21. Vector according to claim 20, characterized in that it comprises the elements allowing the expression and/or secretion of the said sequences in a host cell.

22. Host cell transformed by a vector according to one of claims 20 and 21.

23. Cell according to claim 22, characterized in that it is an eukaryotic or prokariotic cell.

24. Mammal, except man, characterized in that it comprises a cell according to claim 22 or 23.

25. Use of a nucleic acid sequence according to one of claims 16 to 18, as a primer or a probe, for the detection and/or amplification of a nucleic acid sequence.

26. Use of a nucleic acid sequence according to one of claims 1 to 12, and 15, for the production of a recombinant or synthetic polypeptide.

27. Method of producing a recombinant polypeptide, characterized in that transformed cells according to one of claims 22 and 23 are cultured under conditions allowing the expression of the said recombinant polypeptide and in that the said recombinant polypeptide is recovered.

28. Recombinant or synthetic polypeptide, characterized in that it is capable of being obtained by a method according to claim 26.

29. Mono- or polyclonal antibodies or fragments thereof, chimeric or immunoconjugated antibodies, characterized in that they are capable of specifically recognizing a polypeptide according to one of claims 13, 14 and 28.

30. Method for screening RNA, cDNA or genomic DNA contained in a biological sample or in libraries, characterized in that it uses a nucleic sequence according to one of claims 16 to 18.

31. Method for the determination of an allelic variability or a loss of heterozygosity, characterized in that it uses a nucleic acid sequence according to one of claims 1 to 12, and 15 to 18.

32. Method for the diagnosis of diabetes and/or pathology related to WRS or correlated with an abnormal expression of a polypeptide having the sequence SEQ ID No. 2, characterized in that one or more antibodies according to claim 29 is(are) brought into contact with the biological material to be tested, under conditions allowing the possible formation of specific immunological complexes between the said polypeptide and the said antibody or antibodies, and in that the immunological complexes possibly formed are detected.

33. Method for determining if a subject is at decrease or increased risk of having diabetes and/or pathology related to WRS comprising the steps of:
a) collecting a biological sample containing genomic DNA or RNA from the subject;
b) determining on at least one gene allele or RNA encoding the protein EIF2AK3, the sequence, or length thereof, of a fragment of said DNA or RNA susceptible of containing a polymorphism associated to a decrease or increased risk of having diabetes and/or pathology related to WRS, fragment which can be amplified by polymerase chain reaction with a set of primers according to one of the claims 16 to 18;
c) observing whether or not the subject is at decrease or increased risk of having diabetes and/or pathology related to WRS by observing if the sequence of said fragment of DNA or RNA contains a polymorphism associated to a decrease or increased risk of having pathology related to WRS, the presence of said polymorphism indicates said subject is at decrease or increased risk of having diabetes and/or pathology related to WRS.

34. Method in vitro for determining if a subject, whose one member of his family is affected by the WRS, is at risk of having WRS comprising the steps of:
a) collecting a biological sample containing genomic DNA or RNA from the subject;
b) determining on the sequence of both alleles of the EIF2AK3 gene, the sequence, or length thereof, of a fragment of said DNA or RNA susceptible of containing a polymorphism associated to the risk of having WRS, fragment which can be amplified by polymerase chain reaction with a set of primers according to one of the claims 16 to 18;
c) observing whether or not the subject is at risk of having WRS by observing if for both alleles, the sequence of said fragment of DNA or RNA carry a mutation associated to a risk of having WRS, the presence of said mutation indicates said subject is at risk of having WRS.

35. Method according to claim 34 for the diagnosis of the risk of having the WRS, characterized in that said polymorphism associated to the risk of having WRS in step b) is the presence of the mutation corresponding to an insertion of a T at position 1103 or a G to A transition at position 1832 in the sequence SEQ ID No. 1, the presence of said mutation on each of the EIF2AK3 gene allele of said subject indicates said subject is at risk of having WRS.

36. Method in vitro for determining if a subject, whose one family's member is affected by the WRS, is at risk of having WRS comprising the steps of:
a) collecting a biological sample containing genomic DNA or RNA from the family's member affected by the WRS and from said subject;
b) determining if the family's member affected by the WRS and said subject present an allelic identity by comparing polymorphic markers which are positioned close to or included in the EIF2AK3 gene, the genotype identity between the family's member affected by the WRS and said subject indicates said subject is at risk of having WRS.

37. Method according to claim 36 for determining if a subject, whose one family's member is affected by the WRS, is at risk of having WRS comprising the steps of:
a) collecting a biological sample containing genomic DNA or RNA from the family's member affected by the WRS and from said subject;
b) determining on the both EIF2AK3 gene alleles of said family's member, the sequence of a fragment of DNA or RNA susceptible of containing a polymorphism associated to the risk of having WRS, fragment which can be amplified by polymerase chain reaction with a set of primers according to one of the claims 16 to 18;
c) determining if the mutation of the sequence of said fragments responsible of the WRS affection identified in step b) is present on the same fragment of both the EIF2AK3 gene alleles of said subject, fragment which can be amplified by polymerase chain reaction with a set of primers according to one of the claims 16 to 18;
d) observing whether or not the subject is at risk of having WRS by observing if the sequence of said fragment on the both EIF2AK3 gene alleles of the subject contains the same mutation as identified in step b) for said family's member, the presence of said mutation on the both alleles indicates said subject is at risk of having WRS.

38. The method according to anyone of claims 33 to 37, wherein the sequence, or length thereof, of a fragment of DNA or RNA susceptible of containing said polymorphism is obtained in step b) by determining the size of and/or sequencing the amplified products obtained after polymerase chain reaction, eventually after a step of reverse transcription.

39. A method according to claim 33, characterized in that said method further comprises a second method for assaying a biological sample from said subject for levels of at least an additional marker associated with the decreased or increased risk of developing diabetes and/or pathology related to the WRS, the presence of a significantly level of said at least one marker allowing to confirm if said subject is at decreased or increased risk of developing said diabetes and/or pathology related to the WRS.

40. Kit for determining if a subject is at decreased or increased risk of having diabetes and/or pathology related to the WRS, comprising at least one pair of primers capable of amplifying a fragment of genomic DNA or RNA encoding the protein EIF2AK3 and susceptible of containing a polymorphism associated to a decreased or increased risk of having diabetes and/or pathology related to the WRS, said primers being chosen among the primers according to one of the claims 16 to 18.

41. Kit according to claim 40 characterized in that said kit further comprises means for assaying a biological sample from said subject for levels of at least an additional marker associated with is a decreased or increased risk of having diabetes and/or pathology related to the WRS.

42. Kit according to claim 41, characterized in that said additional associated marker is an additional marker associated with the increased risk of having diabetes and/or pathology related to WRS.

43. Kit for determining if a subject is at risk of having WRS, comprising at least one pair of primers capable of amplifying a fragment of genomic DNA containing a polymorphic marker which is positioned close to or included in the EIF2AK3 gene.

44. Kit for determining if a subject is at risk of having WRS, comprising at least one pair of primers capable of amplifying a fragment of EIF2AK3 genomic DNA susceptible to contain an insertion of a T at position 1103 or a G to A transition at position 1832 in the sequence SEQ ID No. 1, said primers being chosen among the primers according to one of the claims 16 to 18.

45. Method according to anyone of claims 32, 33 and 39 or kit according to anyone of claims 40 to 42, characterized in that said diabetes and/or pathology related to WRS is selected from the group consisting of type 1 diabetes, type 2 diabetes, the others forms of diabetes, osteoporosis, arthritis, hepatic dysfunction, nephropathies and other renal dysfimction and mental retardation.

46. Method according to anyone of claims 32, 33 and 39 or kit according to anyone of claims 40 to 42, wherein the said diabetes and/or pathology related to WRS is selected from the group consisting of type 1 diabetes, type 2 diabetes and the other forms of diabetes.

47. Method according to anyone of claims 32, 33 and 39 or kit according to anyone of claims 40 to 42, wherein said diabetes and/or pathology related to WRS is type 1 diabetes.

48. Use of cell according to one of claims 22 and 23, of a mammal according to claim 25, or of a polypeptide according to one of claims 13 to 14 and 28, for studying the expression or the activity of the EIF2AK3 protein, and the direct or indirect interactions between said EIF2AK3 protein and chemical or biochemical compounds which may be involved in the activity of said EIF2AK3 protein.

49. Use of cell according to one of claims 22 and 23, of a mammal according to claim 25, or of a polypeptide according to one of claims 13, 14 and 28, for screening chemical or biochemical compounds capable of interacting directly or indirectly with the EIF2AK3 protein, and/or capable of modulating the expression or the activity of said EIF2AK3 protein.

50. Method for selecting a chemical or biochemical compound capable of interacting, directly or indirectly, with the EIF2AK3 protein, and/or allowing the expression or the activity of the said EIF2AK3 protein to be modulated, characterized in that it uses a cell according to one of claims 22 and 23, of a mammal according to claim 25, or of a polypeptide according to one of claims 13, 14 and 28.

51. Compound characterized in that it is selected by a method according to claim 50.

52. Compound according to claim 51, characterized in that it allows:
a modulation of the level of EIF2AK3 protein expression; and/or
an increase of pancreatic β-cells or integrity thereof; and/or
the prevention or treatment of diabetes and/or pathology related to the WRS.

53. Compound according to one of claims 51 and 52, characterized in that it is chosen from:
a) an antibody according to claim 29;
b) a polypeptide according to one of claims 13, 14 and 28;
c) a vector according to either of claims 20 and 21;
d) a sense or anti-sense nucleic sequence according to claim 19.

54. Compound according to one of claims 51 to 53, as a medicament.

55. Compound according to claim 54, for the prevention and/or treatment of diabetes and/or pathology related to WRS.

56. Compound according to claim 55, characterized in that said diabetes and/or pathology related to WRS is selected from the group consisting of type 1 diabetes, type 2 diabetes, the others forms of diabetes, osteoporosis, arthritis, hepatic dysfunction, nephropathies or other renal dysfunction, mental retardation.

57. Compound according to claim 55, characterized in that said diabetes and/or pathology related to VWRS is selected from the group consisting of type 1 diabetes, type 2 diabetes and the others forms of diabetes.

58. Compound according to claim 55, characterized in that said diabetes and/or pathology related to WRS is type 1 diabetes.
